(19) United States
(12) Patent Application Publication
(10) Pub. No.: US 2012/0295347 A1
(43) Pub. Date: Nov. 22, 2012

(54) Methods and Compositions for Producing Endothelial Progenitor Cells from Pluripotent Stem Cells

(76) Inventor: Steven Kessler, Belmont, CA (US)

(21) Appl. No.: 13/477,002

(22) Filed: May 21, 2012

Related U.S. Application Data
(60) Provisional application No. 61/488,319, filed on May 20, 2011.
(60) Provisional application No. 61/496,436, filed on Jun. 13, 2011.

(51) Int. Cl. C12N 5/071; C12N 5/0735

(52) U.S. Cl. 435/366; 435/377; 435/325

(57) ABSTRACT

Aspects of the present invention are drawn to methods and compositions for producing endothelial progenitor cells (EPCs) in vitro from pluripotent stem cells and compositions containing such EPCs. The methods produce sufficient EPCs to use in therapeutic applications. In certain embodiments the EPCs are bipotent, giving rise to both vascular and lymphatic endothelial cells. In certain embodiments, EPCs express one or more of the following gene products: LYVE-1, PV-1/PAL-E, CD31, and CD34.

This application claims priority to U.S. Provisional Application No. 61/488,319 filed on May 20, 2011 and U.S. Provisional Application No. 61/496,436 filed on Jun. 13, 2011, both of which are incorporated by reference in their entirety.

### INTRODUCTION

The use of embryonic stem cells (ES cells) and other pluripotent stem cells (e.g., induced pluripotent stem cells (iPS cells)) in tissue engineering and other applications holds great promise for advancing cell-based therapies. Developing methods for deriving specific types of tissue progenitor or more mature cells from these pluripotent cell types that can then be employed to study, diagnose, or target disease is thus an active area of investigation. One such area of investigation is the formation of blood and lymphatic vasculature by endothelial progenitor cells (EPCs). Damage to or dysregulation of vasculature is involved in the pathogenesis of a wide array of diseases. It has been estimated that therapeutics targeting blood vessel formation could benefit more than 500 million people (Carmeliet, (2005) Nature 438:932-6).

EPCs are immature endothelial cells (ECs), which have the capacity to proliferate, migrate, and differentiate into endothelial cells but have not yet acquired the full characteristics of more mature cells. They may participate in the formation of new blood vessels by recruitment from bone marrow or other sites to new sites of de novo differentiation to ECs (“vasculogenesis”), or by sprouting from pre-existing vessels (“angiogenesis”). A variety of antigens/antigen combinations have been postulated as markers of EPCs (see, e.g., Watt et al., J.R. Soc. Interface (2010) 7, S731-S751; incorporated herein by reference).

In principle, EPCs or their products could be used to revascularize and help repair or regenerate damaged tissue. For example, EPCs have been shown to significantly participate in constructing endothelium of new vessels in situations of tissue regeneration such as burns, bypass coronary artery grafting, and acute myocardial infarction. In these instances, bone marrow-derived EPCs are recruited to the blood circulation and home to injured and regenerating tissues where they participate in the formation of new blood vessels. Increased circulating EPC levels correlate with improved outcomes in cardiovascular and cerebrovascular ischemia (see, e.g., Sobrino et al. (2007) Stroke 38:2759-2764.).

In addition, EPCs could be used to target sites of pathological vascularization such as in tumor progression, various forms of autoimmunity and macular degeneration. For these applications, the functions of the EPCs could be engineered further, such as to improve homing or targeting efficiency in vivo, modify the actions of drugs, or to produce growth stimulating or inhibiting factors. For example, EPCs can be aimed with therapeutic payloads protected within the cells, and once they have homed to a tumor they can be triggered to induce cell death in surrounding tumor cells (Debatin et al. (2008) Gene Therapy 15: 780-786).

However, EPCs are not always abundant in either circulating blood or the bone marrow. In fact, low abundance of EPCs represents one of the critical issues to overcome in the clinical application of EPCs (see Kawamoto et al. (2007) Catheterization and Cardiovascular Interventions 70:477-484). Increased EPC levels in the clinic currently can be achieved by transplantation from a donor, which involves isolating and expanding EPCs from a donor followed by transplanting the expanded EPCs to the recipient.

As an alternative to obtaining and expanding EPCs from a subject, EPCs for clinical applications can be obtained by directed differentiation of pluripotent stem cells, including human embryonic stem cells (see, e.g., James et al. 2010 Nat. Biotechnology 28(2): 161-166). Aspects of the present invention are drawn to generating sufficient EPCs from pluripotent stem cells for therapeutic applications.

### SUMMARY

Aspects of the present invention are drawn to methods and compositions for producing endothelial progenitor cells (EPCs) in vitro from pluripotent stem cells and compositions containing such EPCs. The methods produce sufficient EPCs to use in therapeutic applications. In certain embodiments the EPCs are bipotent, giving rise to both vascular and lymphatic endothelial cells. In certain embodiments, EPCs express one or more of the following genes: LYVE-1, PV-1, CD31, and CD34.

In certain embodiments the invention provides a method of differentiating stem cells, such as human embryonic stem cells, into an endothelial progenitor cell comprising a) forming an embryoid body from the stem cells; b) transferring the embryoid body from a first tissue culture vessel to a second tissue culture vessel; c) contacting the embryoid body of b) with a first differentiation cocktail; d) plating the embryoid body of c) on an adherent surface so as to form a cell monolayer; e) contacting the cell monolayer of d) with a second differentiation cocktail; f) contacting the cell monolayer off) with a TGF-β inhibitor; g) continuing to culture the cell monolayer until endothelial progenitors appear. In some embodiments the endothelial progenitors are proliferating in cell culture. In some embodiments the endothelial progenitor cell is a bipotential cell, e.g., a cell having the capability to differentiate into vascular endothelial cells and lymphatic endothelial cells. In some embodiments the adherent surface is coated with a matrix such as one or more extracellular matrix proteins, e.g. fibronectin. In some embodiments the TGF-β inhibitor may be SB431542. In some embodiments the cells may be cultured for about 7 days after the cells are contacted with the TGF-β inhibitor.

In some embodiments the invention provides a method of differentiating embryonic stem cells into an endothelial progenitor cell comprising a) forming an embryoid body from the embryonic stem cells; b) culturing the embryoid body; c) contacting the embryoid body with a differentiation cocktail comprising BMP4 and optionally comprising activin; d) contacting the embryoid body of c) with a differentiation cocktail comprising activin and BMP4; e) contacting the embryoid body of d) with FGF2; f) transferring the embryoid body of e) to an adherent cell culture vessel so as to form a cell monolayer; g) contacting the cell monolayer off) with differentiation cocktail comprising BMP4, FGF2 and VEGF; h) contacting the cell monolayer of g) with a TGF-β inhibitor and a cell culture media lacking exogenously added BMP4; i) culturing the monolayer of g) for a sufficient period of time to obtain endothelial progenitor cells, thereby differentiating embryonic stem cells into an endothelial progenitor cell.

In other embodiments the invention provides a method of differentiating cells expressing TERT, OCT4, SSEA4 and TRA-160 into an endothelial progenitor cell comprising a) forming an embryoid body from the cells expressing TERT, OCT4, SSEA4 and TRA-160; b) culturing the embryoid body; c) contacting the embryoid body with a differentiation cocktail comprising BMP4 and optionally comprising activin; d) contacting the embryoid body of c) with a differentiation cocktail comprising activin and BMP4; e) contacting the embryoid body of d) with FGF2; f) transferring the embryoid body of e) to an adherent cell culture vessel so as to form a cell monolayer; g) contacting the cell monolayer off) with differentiation cocktail comprising BMP4, FGF2 and VEGF; h) contacting the cell monolayer of g) with a TGF-β inhibitor and a cell culture media lacking exogenously added BMP4; i) culturing the monolayer of g) for a sufficient period of time to obtain endothelial progenitor cells, thereby differentiating cells expressing TERT, Oct4, SSEA4 and TRA-160 into an endothelial progenitor cell.

In some embodiments the invention provides a method of differentiating embryonic stem cells into an endothelial tip cell comprising a) forming an embryoid body from the embryonic stem cells; b) culturing the embryoid body; c) contacting the embryoid body with a differentiation cocktail comprising BMP4 and optionally comprising activin; d) contacting the embryoid body of c) with a differentiation cocktail comprising activin and BMP4; e) contacting the embryoid body of d) with FGF2; f) transferring the embryoid body of e) to an adherent cell culture vessel so as to form a cell monolayer; g) contacting the cell monolayer off) with differentiation cocktail comprising BMP4, FGF2 and VEGF; h) contacting the cell monolayer of g) with a TGF-β inhibitor and a cell culture media lacking exogenously added BMP4; i) culturing the monolayer of g) for a sufficient period of time to obtain endothelial tip cells, thereby differentiating embryonic stem cells into an endothelial tip cell.

In other embodiments the invention provides a method of differentiating cells expressing TERT, OCT4, SSEA4 and TRA-160 into an endothelial tip cell comprising a) forming an embryoid body from the cells expressing TERT, OCT4, SSEA4 and TRA-160; b) culturing the embryoid body; c) contacting the embryoid body with a differentiation cocktail comprising BMP4 and optionally comprising activin; d) contacting the embryoid body of c) with a differentiation cocktail comprising activin and BMP4; e) contacting the embryoid body of d) with FGF2; f) transferring the embryoid body of e) to an adherent cell culture vessel so as to form a cell monolayer; g) contacting the cell monolayer off) with differentiation cocktail comprising BMP4, FGF2 and VEGF; h) contacting the cell monolayer of g) with a TGF-β inhibitor and a cell culture media lacking exogenously added BMP4; i) culturing the monolayer of g) for a sufficient period of time to obtain endothelial tip cells, thereby differentiating cells expressing TERT, Oct4, SSEA4 and TRA-160 into an endothelial tip cell.

In some embodiments the invention provides a method of differentiating embryonic stem cells into a cell expressing Delta-like 4 (Dll4) protein comprising a) forming an embryoid body from the embryonic stem cells; b) culturing the embryoid body; c) contacting the embryoid body with a differentiation cocktail comprising BMP4 and optionally comprising activin; d) contacting the embryoid body of c) with a differentiation cocktail comprising activin and BMP4; e) contacting the embryoid body of d) with FGF2; f) transferring the embryoid body of e) to an adherent cell culture vessel so as to form a cell monolayer; g) contacting the cell monolayer off) with differentiation cocktail comprising BMP4, FGF2 and VEGF; h) contacting the cell monolayer of g) with a TGF-β inhibitor and a cell culture media lacking exogenously added BMP4; i) culturing the monolayer of g) for a sufficient period of time to obtain cells expressing Dll4, thereby differentiating embryonic stem cells into cells expressing Dll4.

In other embodiments the invention provides a method of differentiating cells expressing TERT, OCT4, SSEA4 and TRA-160 into a cell expressing Dll4 comprising a) forming an embryoid body from the cells expressing TERT, OCT4, SSEA4 and TRA-160; b) culturing the embryoid body; c) contacting the embryoid body with a differentiation cocktail comprising BMP4 and optionally comprising activin; d) contacting the embryoid body of c) with a differentiation cocktail comprising activin and BMP4; e) contacting the embryoid body of d) with FGF2; f) transferring the embryoid body of e) to an adherent cell culture vessel so as to form a cell monolayer; g) contacting the cell monolayer off) with differentiation cocktail comprising BMP4, FGF2 and VEGF; h) contacting the cell monolayer of g) with a TGF-β inhibitor and a cell culture media lacking exogenously added BMP4; i) culturing the monolayer of g) for a sufficient period of time to obtain Dll4 expressing cells, thereby differentiating cells expressing TERT, Oct4, SSEA4 and TRA-160 into a cell expressing Dll4.

In further embodiments the invention provides a method of differentiating a human embryonic stem (hES) cell into an endothelial progenitor cell comprising a) forming an embryoid body from the hES cells under conditions that promote the formation of uniformly sized embryoid bodies; b) culturing the embryoid body of a) for about a day; c) contacting the embryoid body of b) with BMP4 and optionally activin A; d) transferring the embryoid body of c) to a low attachment culture vessel; e) contacting the embryoid body of d) with BMP4 and activin A; f) culturing the embryoid body of e) for about a day; g) contacting the embryoid body off) with FGF2; h) culturing the embryoid body of g) for about 2 days; i) disaggregating the embryoid body into smaller clumps of cells; j) plating the smaller clumps of cells in an adherent tissue culture vessel coated with fibronectin; k) contacting the plated cells of j) with BMP4, FGF2 and VEGF; 1) culturing the cells of k) for about 3 days; m) transferring the cell monolayer of 1) to a tissue culture vessel without a matrix and contacting the cells of l) with S13431542 and a media without any exogenously added BMP4; n) culturing the cells of m) for about 7 days thereby differentiating hES cells into endothelial progenitor cells.

In other embodiments the invention provides a method of differentiating cells expressing TERT, Oct4, SSEA4 and TRA-160 into an endothelial progenitor cell comprising a) forming an embryoid body from the cells expressing TERT, Oct4, SSEA4 and TRA-160 under conditions that promote the formation of uniformly sized embryoid bodies; b) culturing the embryoid body of a) for about a day; c) contacting the embryoid body of b) with BMP4 and optionally activin A; d) transferring the embryoid body of c) to a low attachment culture vessel; e) contacting the embryoid body of d) with BMP4 and activin A; f) culturing the embryoid body of e) for about a day; g) contacting the embryoid body off) with FGF2; h) culturing the embryoid body of g) for about 2 days; i) disaggregating the embryoid body into smaller clumps of cells; j) plating the smaller clumps of cells in an adherent tissue culture vessel coated with fibronectin; k) contacting the plated cells of j) with BMP4, FGF2 and VEGF; l) culturing the cells of k) for about 3 days; m) transferring the cell monolayer of 1) to a tissue culture vessel without a matrix and contacting the cells of l) with SB431542 and a media without any exogenously added BMP4; n) culturing the cells of m) for about 7 days thereby differentiating cells expressing TERT, Oct4, SSEA4 and TRA-160 into endothelial progenitor cells.

In certain embodiments, the endothelial progenitor cells made by the methods described infra are bipotential endothelial progenitor cells, e.g. progenitor cells that can differentiate into vascular endothelial cells and lymphatic endothelial cells. In certain embodiments the cells made by the methods described infra express both LYVE-1 and PV-1PAL-E. In other embodiments the cells made by the methods described infra comprise a population of cells comprising cells expressing LYVE-1 and cells expressing PV-1PAL-E.

In other embodiments the invention provides a proliferating in vitro cell population comprising bipotential endothelial progenitor cells, wherein the cell population comprises cells that can differentiate into both lymphatic endothelial cells and vascular endothelial cells. The cells may be human cells. The cells may be the progeny of a pluripotent human stem cell.

In still further embodiments the invention provides a proliferating in vitro cell population comprising endothelial tip cells, wherein the endothelial tips can form endothelial tube branches such as vascular and capillary tubes. The endothelial tip cells may be human cells. The endothelial tip cells may be the in vitro progeny of a human pluripotent stem cell.

In still other embodiments the invention provides a proliferating in vitro cell population comprising cells expressing Dll4. The cells expressing Dll4 may be human cells. The cells expressing Dll4 may be the in vitro progeny of a human pluripotent stem cell.

In still other embodiments the invention provides a proliferating cell that expresses both LYVE-1 and PV-1PAL-E.

In further embodiments the invention provides a system for making endothelial cells comprising a first population of cells comprising pluripotent stem cells and a second population of cells comprising bipotential endothelial progenitor cells.

In yet other embodiments the invention provides a system for making endothelial cells comprising a first population of cells expressing the markers TERT, OCT4, SSEA4 and TRA-160 and a second population of cells comprising bipotential endothelial progenitor cells.

In still further embodiments the invention provides a system for making endothelial tip cells comprising a first population of cells comprising pluripotent stem cells and a second population of cells comprising bipotential endothelial progenitor cells. The endothelial tip cells may have the ability to sprout new endothelial tubules such as vascular endothelial tubules and capillary endothelial tubules.

In still further embodiments the invention provides a system for making endothelial tip cells comprising a first population of cells comprising cell expressing the markers TERT, OCT4, SSEA4 and TRA-160 and a second population of cells comprising endothelial tip cells. The endothelial tip cells may have the ability to sprout new endothelial tubules such as vascular endothelial tubules and capillary endothelial tubules.

In other embodiments the invention provides a system for making cells expressing Dll4 comprising a first population of cells comprising pluripotent stem cells and a second population of cells comprising cells expressing Dll4.

In further embodiments the invention provides a first and second cell population comprising a first population of cells comprising pluripotent stem cells and a second population of cells comprising endothelial progenitor cells.

In still other embodiments the invention provides a first and second cell population comprising a first population of cells expressing the markers TERT, OCT4, SSEA4 and TRA-160 and a second population of cells comprising endothelial progenitor cells, In further embodiments the invention provides a first and second cell population comprising a first population of cells comprising pluripotent stem cells and a second population of cells comprising endothelial tip cells.

In still further embodiments the invention provides a first and second cell population comprising a first population of cells comprising pluripotent stem cells and a second population of cells comprising cells expressing Dll4.

In other embodiments the invention provides a first and second cell population comprising a first population of cells comprising cell expressing the markers TERT, OCT4, SSEA4 and TRA-160 s and a second population of cells comprising cells expressing Dll4.

### BRIEF DESCRIPTION OF THE DRAWINGS

FIG. 1: Exemplary protocol for EPC production from hESCs.

FIG. 2: Exemplary timeline for addition of soluble factors and growth conditions for EPC production from hESCs.

FIG. 3: Embryoid Body (EB) formation in AggreWell™ 400 plates.

FIG. 4: EBs harvested from AggreWells™ at 24 Hours.

FIGS. 5A and 5B: Description of markers of endothelial cells that are shared with other cell types.

FIG. 6: Metrics for Medium-Scale EPC Production.

FIG. 7: FACS Analysis of Cell Fractions as described in FIG. 6. Percentages of positive cells for CD31 and CD34 are shown for each of the cell lines listed before separation (unseparated), or after CD34 enrichment (purification), or after CD34 depletion.

FIG. 8: Analysis of Different Fractions of EPC-differentiated H9 Cells. Percent positive cells in the population and mean fluorescence intensity (MFI) are shown for CD31 and CD34 cell markers. Differentiated H9 cells were analyzed as unseparated cells, positively selected for CD31, or negatively selected for CD31.

FIG. 9: Analysis of Different Fractions of EPC-differentiated ESI 017 Cells. Percent positive cells in the population and mean fluorescence intensity (MFI) are shown for CD31 and CD34 cell markers. EPC-differentiated ESI 017 cells were analyzed as unseparated cells, positively selected for CD34, or CD34 depleted.

FIG. 10: Analysis of Different Fractions of EPC-differentiated H9 Cells. Percent positive cells in the population and mean fluorescence intensity (MFI) are shown for CD31 and CD34 cell markers. EPC-differentiated H9 cells were analyzed as unseparated cells, positively selected for CD34, or CD34 depleted.

FIG. 11: Analysis of Different Fractions of EPC-differentiated H1 Cells. Percent positive cells in the population and mean fluorescence intensity (MFI) are shown for CD31 and CD34 cell markers. EPC-differentiated H1 cells were analyzed as unseparated cells, positively selected for CD34 (using either Dynabeads or MACS Microbeads), or CD34 depleted (using either Dynabeads or MACS Microbeads).

FIG. 12: Analysis of Different Fractions of EPC-differentiated ESI 035 Cells. Percent positive cells in the population and mean fluorescence intensity (MFI) are shown for CD31 and CD34 cell markers. EPC-differentiated ESI 035 cells were analyzed as unseparated cells, positively selected for CD34, or CD34 depleted.

FIG. 13: LYVE-1 expression data from microarray analysis of EPCs positively or negatively selected for CD31 expression. LYVE-1 RNA expression is positively correlated with CD31 antigen expression (see Examples section below). M+SP: CD31 positively selected cells cultured on tissue culture treated plastic surface; M+SM: CD31 positively selected cells cultured on a layer of Matrigel; M+EGM: CD31 positively selected cells cultured in complete EGM-2 medium. D+SP: CD31 positive cells sorted from CD31 depleted fraction and further cultured on tissue culture treated plastic; D-SP: CD31 negative cells sorted from CD31 depleted fraction and further cultured on tissue culture treated plastic; HMVEC P6: Human microvascular endothelial cells, passage 6; HUVEC: human umbilical vascular endothelial cells at passage 6 (P6) and without passage (w/o P6).

FIG. 14: Shows different 3 day recovery protocols for culturing thawed EPCs derived from 3 hESC lines and cryopreserved on days 14-15 of the derivation process (listed at top).

FIG. 15: Shows cell counts for 3 day recovery protocols of FIG. 14.

FIG. 16: Shows FACS plots of CD34 vs. LYVE-1 expression on cells in FIG. 15.

FIG. 17: Shows FACS plots of PV-1/PAL-E vs. CD34 FACS expression on cells in FIG. 15.

FIG. 18: Shows FACS plots of PV-1/PAL-E vs. LYVE-1 FACS expression on cells in FIG. 15.

FIG. 19: Shows different 10 Day expansion protocols for culturing thawed EPCs derived from ESI 035 and cryopreserved on day 15 of the derivation process.

FIG. 20: Shows cell counts for 10 day expansion protocols of FIG. 19.

FIG. 21: Shows different protocols for 7 day expansion of thawed EPCs derived from 2 hESC lines and cryopreserved on days 14-15 of the derivation process (listed at top).

FIG. 22: Shows cell counts for 7 day expansion protocols of FIG. 21.

FIG. 23: Shows endothelial-like morphology of day 15 EPCs derived from ESCs under chemically-defined, serum free conditions.

FIGS. 24A and 2413: FIG. 24A shows antigen expression on day 15 CD31-immunoselected EPCs derived from WA09 (H9) ESCs. FIG. 24B shows a time course of antigen expression on day 15, day 21, and day 36 CD31-immunoselected EPCs derived from WA09 (H9) ESCs.

FIG. 25: Shows CD31 and CD34 expression on various CD34 immunoselected fractions from EPCs derived from 4 different ESC lines.

FIG. 26: Shows a heatmap of gene expression from microarray analysis of consecutive, medium-scale EPC derivations from different ECS lines.

FIG. 27: Shows the organization of H1 ESC-derived EPCs into microvessels after co-implantation with HT1080 fibrosacroma cells into NOD/SCID mice.

FIG. 28a-p is a histogram showing expression of a number of endothelial cell associated genes.

FIG. 29 shows co-expression of Dll4 and CD34 on cryopreserved EC cells which were thawed and cultured overnight.

FIG. 30 is a photo micrograph showing the spontaneous formation of tubular or capillary-like structures by hESC-derived EPCs.

### DEFINITIONS

The term “embryonic stem cells” (ES cells) or “human embryonic stem cells” (hES cells) refers to cells derived from the inner cell mass of blastocysts, blastomeres, or morulae that have been serially passaged as cell lines while maintaining an undifferentiated state (e.g. expressing TERT, OCT4, and SSEA and TRA antigens specific for ES cells of the species). The ES cells may be derived from fertilization of an egg cell with sperm or DNA, nuclear transfer, parthenogenesis, or by means to generate hES cells with hemizygosity or homozygosity in the MHC region. While ES cells have historically been defined as cells capable of differentiating into all of the somatic cell types as well as germ line when transplanted into a preimplantation embryo, candidate ES cultures from many species, including human, have a more flattened appearance in culture and typically do not contribute to germ line differentiation, and are therefore called “ES-like cells.” It is commonly believed that human ES cells are in reality “ES-like”, however, in this application we will use the term ES cells to refer to both ES and ES-like cell lines. Exemplary patents describing ES cells, including primate/human ES cells, include U.S. Pat. Nos. 7,582,479; 7,217,569; 6,887,706; 6,602,711; 6,280,718; and 5,843,780 to Thomson (each of which is incorporated by reference herein in its entirety).

The term “embryo-derived” (“ED”) cells (or “human embryo derived cells”; hED cells) refers to blastomere-derived cells, morula-derived cells, blastocyst-derived cells including those of the inner cell mass, embryonic shield, or epiblast, or other totipotent or pluripotent stem cells of the early embryo, including primitive endoderm, ectoderm, and mesoderm and their derivatives, but excluding hES cells that have been passaged as cell lines. The ED cells may be derived from fertilization of an egg cell with sperm or DNA, nuclear transfer, chromatin transfer, parthenogenesis, analytical reprogramming technology, or by means to generate ES cells with hemizygosity or homozygosity in the HLA region.

The term “embryonic germ cells” (EG cells) (or “human embryonic germ cells” hEG cells) refer to pluripotent stem cells derived from the primordial germ cells of fetal tissue or maturing or mature germ cells such as oocytes and spermatogonial cells, that can differentiate into various tissues in the body. The EG cells may also be derived from pluripotent stem cells produced by gynogenetic or androgenetic means, i.e., methods wherein the pluripotent cells are derived from oocytes containing only DNA of male or female origin and therefore will comprise all female-derived or male-derived DNA (see U.S. application Nos. 60/161,987, filed Oct. 28, 1999; 091697,297, filed Oct. 27, 2000; 09/995,659, filed Nov. 29, 2001; 10/374,512, filed Feb. 27, 2003; PCT application no. PCT/US/00/29551, filed Oct. 27, 2000; the disclosures of which are incorporated herein in their entirety).

The term “iPS cells” or “human iPS cells” refers to cells with properties similar to ES cells, including the ability to form all three germ layers when transplanted into immunocompromised mice wherein said iPS cells are derived from cells of varied somatic cell lineages following exposure to de-differentiation factors, for example hES cell-specific transcription factor combinations: KLF4, SOX2, MYC, and OCT4 or SOX2, OCT4, NANOG, and LIN28. Any convenient combination of de-differentiation factors may be used to produce iPS cells. Said iPS cells may be produced by the expression of these genes through vectors such as retroviral, lentiviral or adenoviral vectors as is known in the art, or through the introduction of the factors as proteins, e.g., by permeabilization or other technologies. For descriptions of such exemplary methods see: PCT application number PCT/US2006/030632, filed on Aug. 3, 2006; U.S. application Ser. No. 11/989,988; PCT Application PCT/US2000/018063, filed on Jun. 30, 2000; U.S. application Ser. No. 09,736,268 filed on Dec. 15, 2000; U.S. application Ser. No. 10/831,599, filed Apr. 23, 2004; and U.S. Patent Publication 20020142397 (application Ser. No. 10/015,824, entitled “Methods for Altering Cell Fate”); U.S. Patent Publication 20050014258 (application Ser. No. 10/910,156, entitled “Methods for Altering Cell Fate”); U.S. Patent Publication 20030046722 (application Ser. No. 10/032,191, entitled “Methods for cloning mammals using reprogrammed donor chromatin or donor cells”); and U.S. Patent Publication 20060212952 (application Ser. No. 11/439,788, entitled “Methods for cloning mammals using reprogrammed donor chromatin or donor cells”) all of which are incorporated herein by reference in their entirety.

The term “analytical reprogramming technology” refers to a variety of methods to reprogram the pattern of gene expression of a somatic cell to that of a more pluripotent state, such as that of an iPS, ES, ED, EC or EG cell, wherein the reprogramming occurs in multiple and discrete steps and does not rely simply on the transfer of a somatic cell into an oocyte and the activation of that oocyte (see U.S. application Nos. 60/332,510, filed Nov. 26, 2001; 10/304,020, filed. Nov. 26, 2002; PCT application no. PCT/US02/37899, filed Nov. 26, 2003; U.S. application No. 60/705,625, filed Aug. 3, 2005; U.S. application No. 60/729,173, filed Aug. 20, 2005; U.S. application No. 60/818,813, filed Jul. 5, 2006, PCT/US06/30632, filed Aug. 3, 2006, the disclosure of each of which is incorporated by reference herein).

As used herein, “embryoid body”, “EB” or “EB cells” typically refers to a morphological structure comprised of a population of cells, the majority of which are derived from embryonic stem (“ES”) cells (or other pluripotent stem cells, e.g., iPS cells) that have undergone differentiation. Under culture conditions suitable for EB formation, ES cells proliferate and form small mass of cells that begin to differentiate. In the first phase of differentiation, usually corresponding, to about days 1-4 of differentiation for humans, the small mass of cells forms a layer of endodermal cells on the outer layer, and is considered a “simple embryoid body.” In the second phase, usually corresponding to about days 3-20 post-differentiation for humans, “complex embryoid bodies” are formed, which are characterized by extensive differentiation of ectodermal and mesodermal cells and derivative tissues. As used herein, the term “embryoid body” or “EB” encompasses both simple and complex embryoid bodies unless otherwise required by context. The determination of when embryoid bodies have formed in a culture of ES cells is routinely made by persons of skill in the art by, for example, visual inspection of the morphology. Floating masses of about 20 cells or more are considered to be embryoid bodies (see. e.g., Schmitt, R., et al. (1991) Genes Dev. 5:728-740; Doetschman, T. C., et al. (1985) J. Embryol. Exp. Morph. 87:27-45). It is also understood that the term “embryoid body,” “EB,” or “EB cells” as used herein encompasses a population of cells, the majority of which being pluripotent cells capable of developing into different cellular lineages when cultured under appropriate conditions. As used herein, the term also refers to equivalent structures derived from primordial germ cells, which are primitive cells extracted from embryonic gonadal regions (see, e.g., Shamblott, et al. (1998) Proc Natl Acad Sci (USA) 95:13726-13731). Primordial germ cells, sometimes also referred to in the art as ES cells or embryonic germ cells, when treated with appropriate factors form pluripotent ES cells from which embryoid bodies can be derived (see, e.g., Hogan, U.S. Pat. No. 5,670,372; Shamblott, et al., supra).

The term “cell expressing gene X”, “gene X is expressed in a cell” (or cell population), or equivalents thereof, means that analysis of the cell using a specific assay platform provided a positive result. The converse is also true (i.e., by a cell not expressing gene X, or equivalents, is meant that analysis of the cell using a specific assay platform provided a negative result). Thus, any gene expression result described herein is tied to the specific probe or probes employed in the assay platform (or platforms) for the gene indicated.

The term “cell line” refers to a mortal or immortal population of cells that is capable of propagation and expansion in vitro.

The term “clonal” refers to a population of cells obtained the expansion of a single cell into a population of cells all derived from that original single cells and not containing other cells.

The term “oligoclonal” refers to a population of cells that originated from a small population of cells, typically 2-1000 cells, that appear to share similar characteristics such as morphology or the presence or absence of markers of differentiation that differ from those of other cells in the same culture. Oligoclonal cells are isolated from cells that do not share these common characteristics, and are allowed to proliferate, generating a population of cells that are essentially entirely derived from the original population of similar cells.

LYVE-1: lymphatic vessel endothelial hyaluronan receptor 1. LYVE-1 is a marker of lymphatic endothelial cells and is a ligand-specific transporter trafficking between intracellular organelles (TGN) and the plasma membrane. This gene plays a role in autocrine regulation of cell growth mediated by growth regulators containing cell surface retention sequence binding (CRS). LYVE-1 may act as an hyaluronan (HA) transporter, either mediating its uptake for catabolism within lymphatic endothelial cells themselves, or its transport into the lumen of afferent lymphatic vessels for subsequent re-uptake and degradation in lymph nodes. (Gene ID: 10894).

PV-1: PAL-E, PLVAP, plasmalemma vesicle-associated protein 1. PV-1/PAL-E is a blood vascular endothelial marker involved in lymphocyte transendothelial migration. This marker is completely absent from lymphatic endothelial cells. (Gene ID: 83483).

### DETAILED DESCRIPTION OF THE INVENTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates as, for example, electronic or print or other formats, which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, aspects of the present invention are drawn to methods and compositions for producing endothelial progenitor cells (EPCs) in vitro and compositions containing such EPCs. Methods for producing EPCs are described first followed by descriptions of compositions comprising such EPCs, including compositions suitable for therapeutic applications.

The present invention provides significant benefits over EPC production protocols currently in use. For example, the percentage of CD31 positive EPCs produced using the present invention prior to a cell purification step (e.g., by FACS sorting or immunomagnetic selection) is increased to 60-99% compared to usually 2-5% in current state of the art protocols (see, e.g., James et al. (2010) Nature Biotechnol. 28(2): 161-166 and Ferreira et al. (2007) Circ. Res. 101:286-294). However, such a purification step is fully compatible with the present invention to further increase the proportion of cells expressing a desired antigen marker. Another benefit of the present invention is that it results in the co-expression of CD34 antigen on most or all CD31 positive cells. This can facilitate the use of currently existing and clinically validated positive selection devices for CD34 already employed for hematopoietic stem and progenitor cell purification and transplantation, thereby expediting the transition of EPCs of the present invention for clinical uses. A further benefit of the present invention is that it can yield EPCs expressing markers of both blood vascular endothelial (e.g., PV-1/PAL-E, PLVAP, plasmalemma vesicle-associated protein 1) and lymphatic endothelial cells (e.g., LYVE-1). This may increase the options for utilizing the cells therapeutically, such as for repairing sites of vascular injury or for targeting sites of tumor angiogenesis (or neovascularization) or metastasis through co-opted lymphatic vessels. A still further benefit of the present invention is that it can yield EPCs with such characteristics in relatively short time periods, e.g., 15 days starting from ES cell cultures, and 10 days starting from 5 day embryoid body cultures seeded into adherent cultures in the examples shown.

The results of the present invention are unlike those obtained using a current state of the art process (see James et al. (2010) ibid.), as indicated from the microarray analysis results therein that suggest less than a 0.5-fold increase in CD34 RNA expression over background in unpurified day 14 endothelial cell cultures (Phase 1-derived cells). Using this process a further increase of 10-fold or more in CD34 RNA expression was observed only as the result of an isolation (i.e., purification) step that increased the CD31-expressing endothelial cells to more than 95%. Also, this reference stated that Phase 1-derived cells do not show increased levels of factors typical of lymphatic endothelial cells.

The results of the present invention are also unlike those obtained using another state of the art process (see Ferreira et al. (2007) ibid.) in which ES cells were grown on mouse embryonic feeders, then differentiated into embryoid bodies for 10 days (the peak day of CD34 expression), then the CD34 positive cells (which varied between around 5-14% of cells depending on the ES cell line of origin) were isolated using immunomagnetic beads, and the cells were then cultured in gelatin-coated dishes in the presence of EGM-2 medium enriched with VEGF-165 and containing fetal bovine serum or knockout serum replacement (KO-SR, which contains bovine albumin). This culture system and process contained numerous materials of xenogeneic origin, including serum or bovine albumin and could reasonably be expected to undergo substantial experimentation and substitutions before it could be translated into clinical therapies. Moreover, this process is comparatively lengthy. Thus, as shown in this reference (Ferreira et al. (2007) ibid.), after seeding the cells isolated from day 10 embryoid bodies, a single passage required 10-15 days, and 3 passages required around 28 days. At this stage the CD34 and CD31 (PECAM1) antigen-expressing cells differentiated from H9 ES cells were 65% and 98% positive, respectively, and from H13 ES cells were 14% and 39% positive, respectively. By comparison, CD34 antigen-expressing cells using the process of the present invention were shown to comprise more than 90%, usually 96-99%, of the cells collected from the third passage at 13 days following seeding from embryoid bodies (18 days from initiation of ES cell culture to form embryoid bodies) in all 3 ES cell lines tested (see Examples). In addition, the process according to the present invention is scalable for producing at least hundreds of millions of EPCs. This significant increase in demonstrated scalability which is not known to have been reported in current state of the art protocols makes the present invention suitable for generating therapeutic amounts of EPCs that can be employed in any of a variety of treatments.

As further detailed below, aspects of the present invention achieve these increases in EPC production by employing one or more of the following: generating uniform EBs; eliminating serum from the EPC generation protocol; using xenogenic component-free conditions in the EPC generating protocol; employing a chemically defined culture system.

In addition, the EPCs generated according to aspects of the present invention are true endothelial progenitor/precursor cells (EPCs) and not committed endothelial cells (ECs), as exemplified by the presence in the population of cells that have stable co-expression of CD34 and CD31 as well as cells that have stable co-expression of LYVE-1 (a lymphatic vessel endothelial cell marker) and PV-1/PAL-E (a blood vascular endothelial cell marker).

### Methods for Producing Endothelial Progenitor Cells (EPCs)

In certain aspects, the subject invention provides methods of producing endothelial progenitor cells (EPCs) from pluripotent stem cells in vitro, e.g., from human embryonic stem cells (hES cells), induced pluripotent stem cells (iPS cells), embryo derived cells (EG cells), embryonic germ cells (EG cells), and the like. Numerous non-limiting embodiments of methods for producing EPCs are provided below, which generally include culturing embryoid bodies (EBs) derived from pluripotent stein cells under mesoderm differentiation conditions followed by culturing the resulting cells in endothelial cell differentiation conditions, thereby producing EPCs. In certain embodiments, the cultures of EPCs are bipotent, meaning that the EPCs individually or EPC cultures have both blood vascular and lymphatic vascular endothelial cell potential (i.e., they can produce blood vascular and/or lymphatic vascular endothelial cells under appropriate differentiation conditions, which may be in vitro, in vivo, or a combination of both). Compositions that find use in methods for producing EPCs from pluripotent stem cells are also described.

In certain embodiments, the methods for producing the EPCs as described herein are performed under chemically defined, scrum-free, xenogenic (i.e., non-human origin) component-free and/or animal-derived component-free conditions. In other words, the compositions obtained by the methods for generating EPCs have not been exposed to serum and/or xenogenic components. Moreover, the EPCs were produced according to the methods described infra without the addition of either serum or a serum substitute such as KOSR and the like. This makes the production methods more consistent and less subject to potential safety concerns by regulatory agencies for therapeutic applications.

### Exemplary EPC Production Protocol

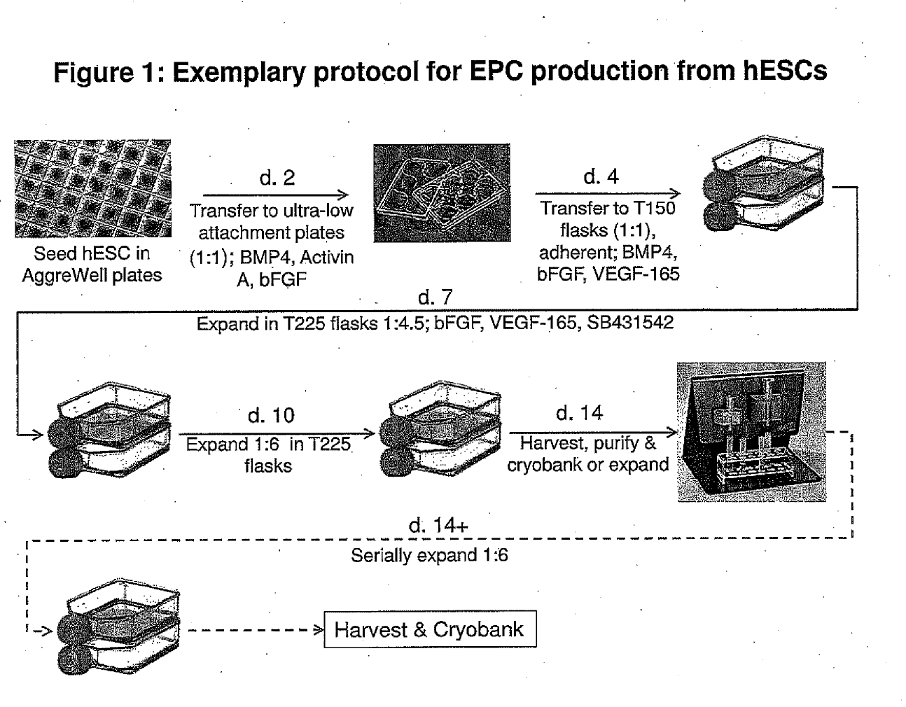

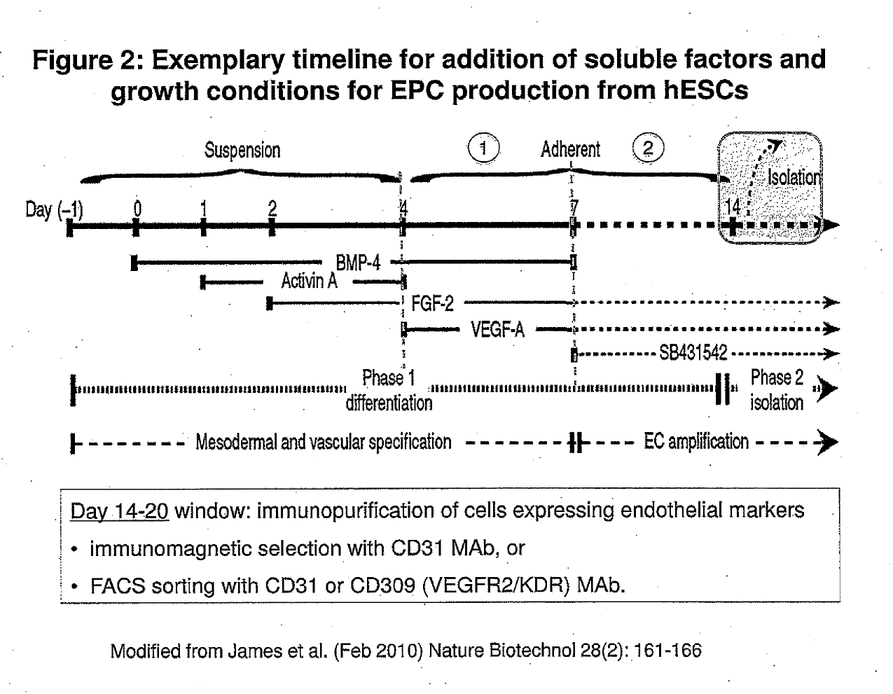

FIG. 1 shows an exemplary EPC production protocol. We emphasize that FIG. 1 and the description in this section are merely exemplary in nature, and thus not meant to limit the scope of the EPC production methods and compositions detailed herein.

In the exemplary production protocol shown in FIG. 1, human ESCs are seeded on day 0 in AggreWell™ plates (Stem Cell Technologies, Vancouver, BC) under conditions that promote the formation of uniformly-sized embryoid bodies (EBs). The AggreWells function by centrifugal forced-aggregation of ESCs into microwells of defined geometry molded into tissue culture plates. The average size of the EBs is controlled by seeding a preset number of ESCs into the microwells (see: AggreWell Technical Manual, version 2.0.0; which can be accessed on the Internet at: http://www(dot)stemcell(dot)com/˜/media/Technical %20Resources/9/29146MAN_{—}2_{—}0_{—}0.ashx). Other methods can be used to similar effect for controlling size and homogeneity of EBs including, without limitation, poly(ethyleneglycol) microwells (Karp et al. (2007) Lab Chip 7:786-794), hydrogel microwells (Hwang et al. (2009) Proc. Natl. Acad. Sci. 106:16978-16983) and microfabricated silicon wafers (Ungrin et al. (2008) PLoS ONE 3(2):e1565); each of which is incorporated herein by reference in its entirety.

On the following day of the protocol (day 1, starting from day 0), bone morphogenetic protein 4 (BMP4) is added to induce differentiation and germ layer specification. Optionally, Activin A is also added to promote mesoderm differentiation. On the next day (day 2) the EBs are transferred to ultra-low attachment 6-well plates and cultured with factors BMP4 and Activin A. Basic fibroblast growth factor (bFGF or FGF-2) are added the following day (day 3). (see, e.g., the order of addition of these factors in FIG. 2; note that the days indicated on the timeline of FIG. 2 are shifted by −1 as compared to FIG. 1). At day 5 of the protocol, the EBs are dissociated and the cells are transferred to adherent cultures in the presence of the cytokines BMP4, bFGF and vascular endothelial growth factor-165 (VEGF-165) in tissue culture flasks (typically T150 or T225 flasks for low to intermediate-scale cell production) The flask may be coated with a matrix, such as an extracellular matrix protein, e.g. fibronectin. At day 8 of the protocol, the cells may be transferred to a flask that does not have a matrix for the cells to grow on (i.e. the cells attach to and grow on the plastic surface of the flask). SB431542 (an inhibitor of TGF-β signaling) is added, and BMP4 is removed (bFGF and VEGF-165 remain), and the culture is split and expanded, further as desired. On or about day 15 the cells are harvested again. Optionally, the cells are purified based on expression of one or more surface membrane antigens, e.g., by positive immunoselection for markers associated with EPCs such as CD34, CD31, VEGFR3, or negative immunoselection for markers associated with other lineages or ESCs, using e.g., immunomagnetic bead selection or fluorescence activated cell sorting (FACS). Optionally, the purified/enriched cells or unpurified cells may then be subjected to quality control analysis or otherwise manipulated as desired (e.g., expanded further in culture, stored (cryobanked), analyzed for gene expression, analyzed by FACS, used in cell therapy, etc., or any combination thereof).

As alluded to above, FIG. 2 provides an example of the sequence of certain culture steps, transfers and cytokine/factor addition based on a protocol for derivation of EPCs described in James et al. (2010 Nat. Biotechnology 28(2): 161-166; incorporated herein by reference in its entirety) but yielding unexpectedly improved results as compared to James et al (ibid). Note that the day count is shifted −1 in FIG. 2 as compared to FIG. 1.

Each of the exemplary process steps and reagents shown in FIGS. 1 and 2 are discussed in further detail below.

Pluripotent Stem Cells

A number of different pluripotent stem cells may be employed in the invention, including ES cells, iPS cells, and any other pluripotent stem cell that can produce EBs in vitro. The pluripotent stem cells can be from a variety of mammalian animals, e.g., primates, bovine, ovine, feline canine, etc. In certain embodiments, the pluripotent stem cells are human.

As noted above, certain aspects of the invention are drawn to the production of EPCs for the therapeutic treatment of a subject (or patient), e.g., treatment of a patient in need of vascular or lymphatic endothelial cells or for use as targeting agent to deliver anti-tumor agents (both of which are discussed below). Thus, in therapeutic embodiments, the EPCs, and thus the pluripotent stem cells from which they are derived, may be autologous to the subject (or patient), non-autologous (allogeneic) to the subject, or a combination thereof.

In embodiments in which the pluripotent stem cells are autologous to the subject, the pluripotent stem cells may be iPS cells generated from cells derived from the patient. In allogeneic settings, the pluripotent stem cells employed may be ES cells or iPS cells, where in certain embodiments the ES or iPS cells are HLA matched to the subject, e.g., similar to HLA matching done for solid organ or bone marrow transplantation. Allogeneic pluripotent stem cells may thus be derived from siblings, other relatives, or non-related individuals that meet certain HLA matching criteria (e.g., are compatible with the subject). The parameters of the specific therapeutic application generally will dictate the amount of HLA matching necessary to provide for an effective therapy. For example, the pluripotent stem cells used to generate EPCs that are used solely as targeting vectors for delivering anti-tumor agents to a specific site (and thus may not have to durably or stably engraft in the subject) can have more relaxed HLA matching criteria as compared to pluripotent stem cells used to generate EPCs for long-term engraftment (e.g., to replace or repair vascular/lymphatic endothelial cell sites in a subject). As such, this aspect of the therapeutic use of EPCs will generally be determined by the user of the subject methods.

Pluripotent stem cells may be obtained from any of a variety of sources, and thus no limitation in this respect is intended. For example, pluripotent stem cells may be obtained from a third party, produced and/or maintained by the user of the subject methods, etc. As such, the description below of pluripotent stem cells is not meant to be limiting.

In some embodiments, the pluripotent stem cells are human ES cells. Exemplary ES cell lines include, but are not limited to, the NIH-registered lines such as WA-01 (H1) and WA-09 (H9) and the ESI lines such as ESI 017, and ESI 035, and ESI051 (Crook et al. (2007) Cell Stem Cell 1:490-494). Methods for the culture, maintenance and propagation of hES cells such that they maintain their pluripotencey have been described and include different combinations of one or more of the following: feeder cells, soluble factors, conditioned media, serum, extracellular matrices, etc. As such, no limitation in this regard ins intended. Examples of conditions for the propagation of hES cells that are more suitable for therapeutic applications include chemically defined media that are feeder cell-free, including, but not limited to:

mTeSR™1 medium (Stem Cell Technologies, Vancouver, BC) on Growth Factor-Reduced Matrigel-coated plates or flasks (Becton-Dickinson Biosciences); TeSR™2 medium (Stem Cell Technologies) on Synthemax (Corning) or Matrigel-coated plates or flasks; X Vivo-10 medium (Lonza) on Synthemax or Matrigel-coated plates or flasks.

Embryoid Bodies in Mesoderm Differentiation Conditions

Methods according to aspects of the present invention include culturing EBs in mesoderm differentiation conditions. The embryoid bodies for use in this culture may be obtained from any number of sources. Thus, while the description below focuses on generating EBs from pluripotent stem cells, such embryoid bodies may be obtained from a third party.

ES cells (and other pluripotent stem cells) have the potential to generate all embryonic cell lineages when they undergo differentiation. Differentiation of ES (including hES cells) can be induced by removing the cells from their adherent culture conditions (e.g., on a feeder cell layer) and growing them in suspension. These differentiation conditions result in the production of an aggregation of ES-derived cells called embryoid bodies (EBs) in which successive differentiation steps occur (see, Itskovitz-Eldor, et al., (2000) Mol Med 6, 88-95). One example of EB formation includes detaching hES cells from their hES cell culture substrate and placed into culture under low-attachment conditions in the absence of FGF-2 (a factor that promotes hES cell self-renewal). Any of a variety of agents can be used for detaching adherent ES cells and EPCs later in the production process, including but not limited to enzymes (such as Accutase, trypsin, dispase, collagenases, etc.) and cation chelating agents (e.g., EDTA).

One exemplary embodiment for large scale production of uniform EBs (which is described in the examples section below) includes seeding substrate-dissociated hES cells in AggreWell plates (Stem Cell Technologies), culturing for 1-2 days, and adding the cytokines BMP4, Activin A, and FGF-2 on each of subsequent days (e.g., days 0, 1 and 2, where day 0 represents the day the BMP was added). The EBs developing in the AggreWell plates can be harvested and transferred to plates/flasks after addition of FGF-2 for the remaining culture interval (i.e., with multiple independent EBs growing in the same culture dish). EBs can be harvested as desired for the next phase of the process.

It is noted that in certain embodiments, all the cytokines used in this (or any other) step of the methods described herein are human recombinant cytokines. In certain embodiments, the human recombinant cytokines are generated in human cells. As detailed and shown in the Examples section, we have found that the use of human recombinant cytokines produced in human cells provides significantly increased yields of EPCs as compared to cytokines that are either non-human recombinant and/or are produced in prokaryotic expression systems. While not being bound by theory, it is though that human recombinant cytokines produced in human cells are more stable during the culture and/or are more effective at activating their cognate receptor due to having post-translational modifications more similar to the in vivo cytokines.

Thus, with respect to the formation of EBs, certain embodiments of the invention include the use of recombinant human BMP, activin A, and/or FGF-2. In certain of these embodiments, the human recombinant BMP, activin A, and/or FGF-2 are produced in human cells.

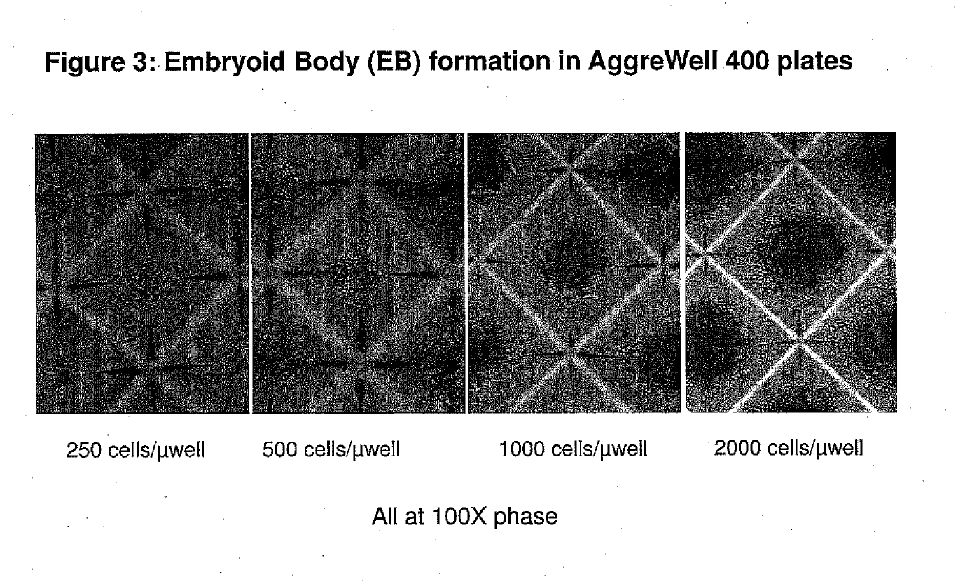

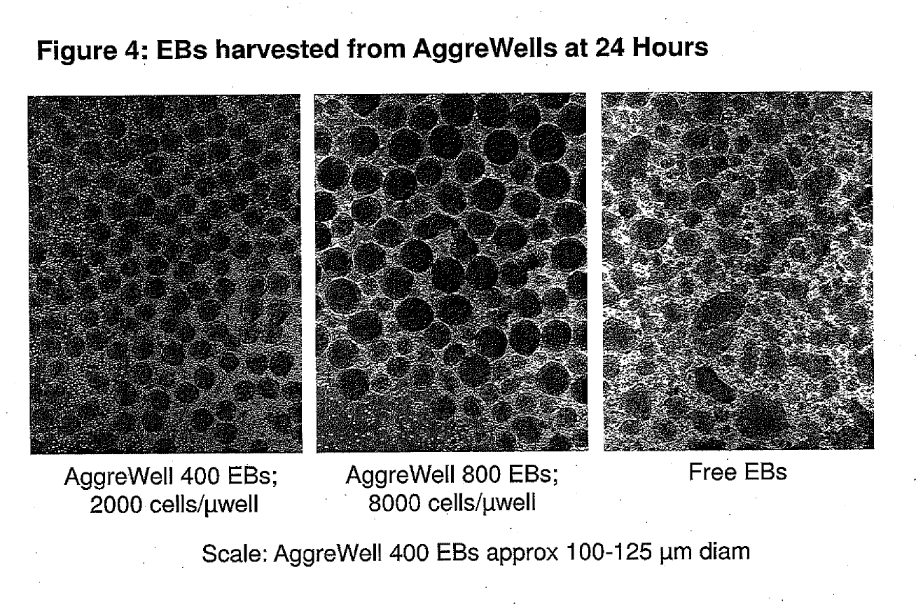

FIG. 3 shows ES cells seeded in AggreWell 400 plates at 250, 500, 1000, and 2000 cells/microwell and the resulting differences in size of the well-demarcated EBs (note that the diameter of each microwell in an AggreWell 400 plate is 400 μm). FIG. 4 shows EBs harvested from an AggreWell 400 plate seeded with 2000 ESCs/microwell and an AggreWell 800 plate seeded with 8000 ESCs/microwell (800 μm diameter microwells) compared to EBs generated free in culture. Note the improved average size homogeneity or uniformity of the EBs produced in the AggreWell plates as compared to the EBs produced free in culture. We have found that improving the size homogeneity of EB production significantly improves the yield of EPCs produced via the methods described herein (see Examples section). Without being bound by theory, it appears that the formation of relatively homogeneous EBs of controlled input cell number and size allows a higher percentage of them to participate in EPC formation in downstream processes, likely due to their more uniform cellular composition, growth and developmental capabilities.

It has been noted above in this application that AggreWell plates may be substituted with other culture systems that allow for the formation of EBs that are more controlled and homogeneous in size (e.g., by forced aggregation of set numbers of ESCs) than those generated free in culture. As such, no limitation in this regard is intended.

hEPC Differentiation and Expansion

Once EBs cultured under mesoderm differentiation conditions are obtained, e.g., as detailed above, they are cultured under endothelial differentiation conditions. In certain embodiments, the endothelial cell differentiation condition is an adherent culture that includes the sequential addition of VEGF and an inhibitor of TGF-β signaling. In certain embodiments, other factors/cytokines may be present in the endothelial differentiation culture (e.g., BMP4 and/or FGF-2).

For example, as EBs may be cultured according to the following endothelial cell differentiation condition (see Examples section for a more detailed description).

EBs are first dissociated (e.g., with Accutase) and then passaged to T flasks coated with matrix components (e.g., extracellular matrix components, synthetic matrices, etc., as are known in the art) that promote cellular attachment (this is the start of the adherent phase). The medium in the adherent culture contains BMP4, FGF-2 and VEGF-165 (e.g., recombinant human cytokines, as detailed above). This culture is allowed to go for about 3 days (72 hours) after which the medium is exchanged with medium containing FGF-2 and VEGF-165 and a TGF-β signaling inhibitor (e.g., SB431542 as described in James et al. 2010, Nature Biotech 28:161). Other TGF-β inhibitors known in the art may also be employed. The cultures can be split and passaged as they approach (but preferably do not reach) confluence. Between days 14-20, the cells in the endothelial cell differentiation culture can be harvested and dissociated using Accutase.

We have noticed that the use of fibronectin as the matrix component at the start of the adherent phase leads to a significant increase in yield of EPCs in the subject methods as compared to using conventional plastic cell culture substrates. Thus, in certain embodiments, the matrix component used on the culture substrate at the start of the adherent phase comprises fibronectin. Later in the adherent phase of the culture (after about day 7), we have found that a different matrix component may be used without significantly impacting EPC yield.

The use of the TGF-β inhibitor has been shown to increase the number and percentage of endothelial cells generated in culture (see James et al. 2010, Nature Biotech 28:161). The culture of cells with the TGFβ inhibitor may proceed for anywhere from 1 to three weeks or longer. The resulting cell population contains EPC at a level of at least 10% or more. In some embodiments, a high percentage and absolute number of EPCs are present in the cell population produced, even without an enrichment step (see Examples section below). For example, EPCs can represent 50% or more of the cells in the population, including 60% or more, 70% or more, 80% or more, 90% or more, up to and including 97% or more, e.g., as defined by surface expression of a variety of antigens associated with these cells, including CD31 and CD34. In addition, the number of EPCs produced can exceed 600 to 800 million in 9-10 days following EB formation in 4 AggreWell plates originally containing around 20 million ESCs each. Thereafter, the EPCs are split 1:6 approximately every 4 days for further expansion as desired. To our knowledge, no EPC production protocol has been described that achieves: 1) the percentage of EPCs described herein without a final cell isolation/enrichment step (e.g., for CD31 expressing cells), and 2) the robust number of EPC produced per input cell. Thus, the EPC production methods described herein are well suited for applications that require large numbers of EPCs, e.g., for cell therapeutic applications.

In certain embodiments, EPCs are subjected to an enrichment process to produce a cellular composition with an increased percentage of EPCs. In certain embodiments, the EPCs may be enriched by isolating cells that are positive for the cell surface expression of CD34 and/or CD31 proteins (i.e., cells that are CD34+ and/or CD31+). A variety of enrichment methods for cell-surface markers (e.g., CD antigens) are known in the art. In general, these enrichment methods employ CD antigen-specific antibodies (i.e., antibodies that bind specifically to CD34 or CD31 under appropriate antibody binding conditions) and a system for selecting the cells to which these antibodies bind. Exemplary enrichment methods/systems include, but are not limited to magenetic-based cell sorting (MACS; Miltenyi), fluorescence activated cell sorting (FACS), panning, etc., each of which is known in the art.

In certain embodiments, EPCs expressing CD34 (CD34+ EPCs) are enriched from the cells produced from the EB/EC culture methods described herein. We have found that CD34 enrichment produces a highly enriched composition of EPCs. Using CD34 as the target antigen for enrichment of EPCs is be advantageous for clinical application of these EPCs as GMP-grade anti-CD34 antibodies and enrichment systems are currently available.

In certain embodiments, the EPCs produced according to the methods of the subject invention are bipotent, meaning that they have developmental potential for both lymphatic and vascular endothelial cells. This bipotent developmental potential may be observed in vivo and/or in vitro.

In certain embodiments, the EPCs produced according to the methods of the subject invention express LYVE-1, which is a known marker for lymphatic endothelial cells (lymph vessels) but is absent in vascular endothelial cells (blood vessels) (see, Fiedler et al., Am. J. Pathology 2006, v. 168). In certain embodiments, the EPCs produced according to the methods of the subject invention express PV-1/PAL-E, which is a known marker for vascular endothelial cells (blood vessels) but is absent in lymphatic endothelial cells (lymph vessels). In certain embodiments, the EPCs produced according to the methods of the subject invention express both LYVE-1 and PV-1/PAL-E. It is noted that populations of EPCs as described herein may include a mixture of EPCs, some of which express either LYVE-1 or PV-1/PAL-E and some that express both markers. Some exemplary distinctions and relationships between these two cell types are described elsewhere (e.g., Oliver et al. (2010) Development 137:363-372 and Bixel et al. (2008) Genes & Development 22:3232-3235, herein incorporated by reference). Given the expression pattern, EPCs produced according to the subject method have bipotent developmental potential, i.e., they can generate (or develop into) both lymphatic and vascular endothelial cells (i.e., can contribute to both lymph and blood vessels).

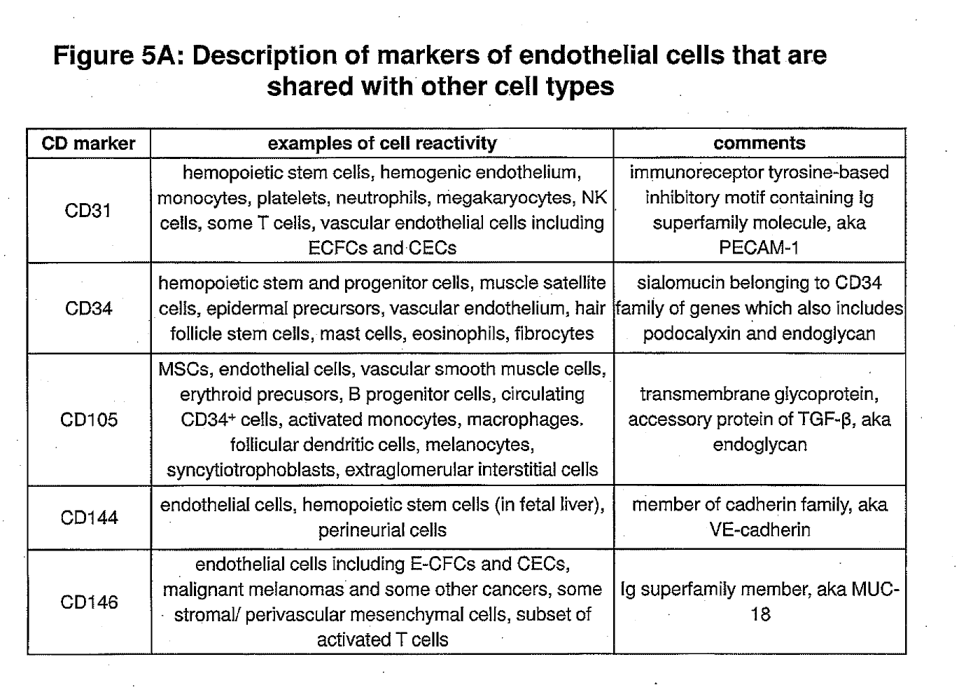

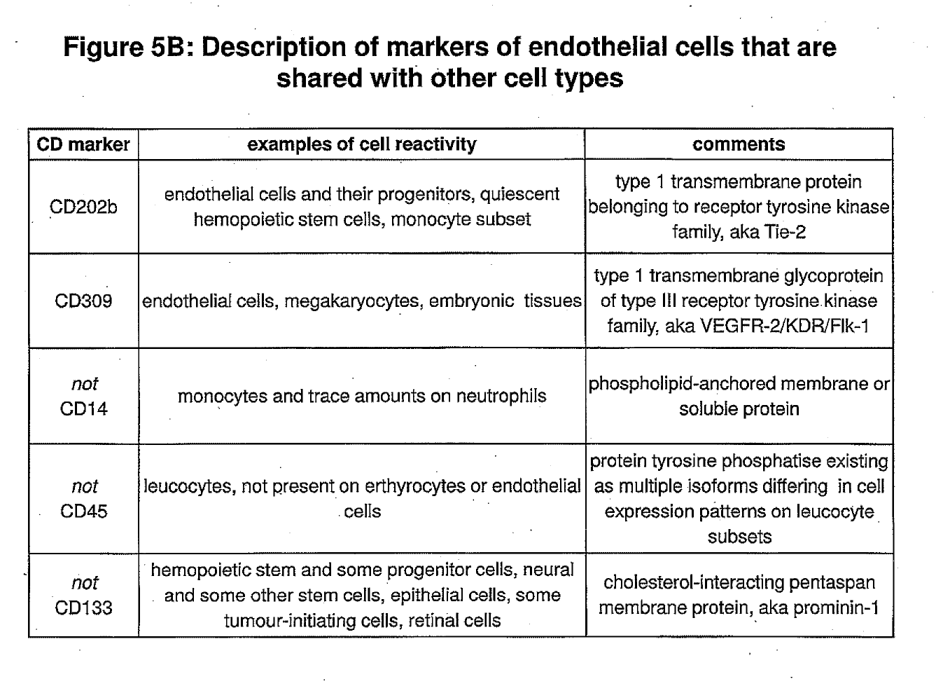

In addition, in certain embodiments, the EPCs produced according to the subject invention exhibit endothelial cell marker expression patterns as described herein (see, e.g., FIGS. 5A and 5B), e.g., being positive for CD31 and/or CD34 expression.

### Compositions Containing Isolated EPCs and Methods of Use

Aspects of the present invention include an isolated population of cells containing endothelial progenitor cells (EPCs). The isolated cell population may be a population in which the EPC are enriched over a starting cell population, i.e., that the EPC are present in number or as a percentage of the cells that is greater than a starting cell population or sample. Cell populations enriched for EPC may be produced using the methods for producing EPC as described herein or by other methods, including by producing and/or isolating the cells from other cell sources, e.g., isolating EPCs from a sample derived from a subject using a cell selection/enrichment process. No limitation to such alternative methods for producing an isolated population containing EPCs is intended.

In certain embodiments, the EPCs in the isolated population are 10% or more of the cells in the population, including 20% or more, 30% or more, 40% or more, 50% or more, 70% or more, 80% or more, 90% or more, 97% or more, up to and include 100% of the cells in the isolated population.

The EPCs of the subject invention can be characterized by their gene expression pattern. In certain embodiments, the EPCs in the isolated population express one or more of (or any combination of) the following genes: LYVE-1, PV-1/PAL-E, CD31, CD34. In certain embodiments, the EPCs in the isolated population express LYVE-1.

The EPCs have the potential to develop into endothelial cells when placed under conditions that promote endothelial cell development, either in vitro or in vivo (and sometimes a combination of both). In certain embodiments, the EPCs are bipotent, meaning that the EPCs can generate both lymphatic and vascular endothelial cells in vitro and/or in vivo.

Aspects of the invention further include compositions containing EPCs as detailed above in a form that is therapeutically useful. As such, the subject invention includes compositions containing isolated EPCs and a pharmaceutically acceptable carrier. By “pharmaceutically acceptable carrier” is meant any composition that can be combined with the isolated EPCs in a manner that is compatible with the therapeutic use of the EPCs. Non-limiting examples of therapeutic uses of EPC as well as pharmaceutically acceptable carriers are provided below (see also, Levenberg et al., US Patent Application Publication 2004/0009589 incorporated herein by reference in its entirety).

Thus, the EPCs of the present invention, or cells derived therefrom, may be used in any of a number of therapeutic applications, e.g., for the repair of blood and/or lymphatic vasculature or as targeting vectors for delivery of therapeutic agents to vascular sites (e.g., tumor vasculature). EPCs can be administered to a subject in any therapeutically acceptable carrier. The subject to which the EPCs (or cells derive therefrom) are administered may have any condition, injury or disease for which EPCs would provide a therapeutic benefit. For example, if a subject has blood vascular cell damage at a specific site, e.g., the heart, the EPCs may be administered to the subject at the site of damage (or in certain embodiments, systemically, where the EPCs, or cells derived therefrom, home to the site of damage).

In certain therapeutic applications, the EPCs may be cultured under endothelial cell-inducing conditions prior to administering the cells to the subject, e.g., to induce endothelial cell production prior to transplantation. For example, EPCs may be induced to form vascular endothelial cells, e.g., on a form or other structure (e.g., a tube), and then be transplanted into a subject at a site in need of endothelial cells. Any convenient endothelial cell producing condition may be employed in such embodiments.

Methods of treatment according to the present invention may also include measuring the rate of generation of endothelial cells at the desired site at one or more time points after transplantation as well as obtaining information as to the performance of the newly formed endothelial cell-containing tissues in the subject. Parameters measured can include the survival, localization, and number of administered cells present at the transplantation site in the patient. The degree cell engraftment or reconstitution may be determined using any of a variety of scanning techniques, e.g., computerized axial tomography (CAT or CT) scan, magnetic resonance imaging (MRI) or positron emission tomography (PET) scans. Functional integration of transplanted cells according to the invention into a subject can be assessed by examining restoration of the function that was damaged or diseased or augmentation of a function associated with the presence of endothelial cells. Cell transplant engraftment, localization and survival can also be done by removing a portion of the target tissue and examining it visually or through a microscope (e.g., in post mortem analysis).

As noted above, EPCs according to the present invention may be combined with a cell support substrate including extracellular matrix components. The substrate may be a gel, for example, Matrigel, from Becton-Dickinson, which is a solubilized basement membrane matrix extracted from the EHS mouse tumor (Kleinman, H. K., et al., Biochem. 25:312, 1986). The primary components of the matrix are laminin, collagen I, entactin, and heparan sulfate proteoglycan (perlecan) (Vukicevic, S., et al., Exp. Cell Res. 202:1, 1992). Matrigel™ also contains growth factors, matrix metalloproteinases (MMPs [collagenases]), and other proteinases (plasminogen activators [PAs]) (Mackay, A. R., et al., BioTechniques 15:1048, 1993). The matrix also includes several undefined compounds (Kleinman, H. K., et al., Biochem. 25:312, 1986; McGuire, P. G. and Seeds, N. W., J. Cell. Biochem. 40:215, 1989), but it does not contain any detectable levels of tissue inhibitors of metalloproteinases (TIMPs) (Mackay, 1993).

In other embodiments, the gel may be a collagen I gel. Such a gel may also include other extracellular matrix components, such as glycosaminoglycans, fibrin, fibronectin, proteoglycans, and glycoproteins. The gel may also include basement membrane components such as collagen IV and laminin. Enzymes such as proteinases and collagenases may be added to the gel, as may cell response modifiers such as growth factors and chemotactic agents.

The EPCs (or endothelial cells derived from the EPCs) either mixed with a gel or simply with a liquid carrier such as PBS, may be injected directly into a tissue site where vasculogenesis is desired. For example, the cells may be injected into ischemic tissue in the heart or other muscle, where the cells will organize into tubules that will anastamose with existing cardiac vasculature to provide a blood supply to the diseased tissue. Other tissues may be vascularized in the same manner. The cells will incorporate into neovascularization sites in the ischemic tissue and accelerate vascular development and anastamosis (see Kawamoto, et al., (2001) Circulation 103, 634-7). It is intended that the cells according to the invention be used to vascularize all sorts of tissues, including connective tissue, muscle tissue, nerve tissue, and organ tissue. Non-blood duct networks may be found in many organs, such as the liver and pancreas, and the techniques of the invention may be used to engineer or promote healing in such tissues as well. For example, embryonic endothelial cells injected into the liver can develop into tubular networks around which native hepatocytes can develop other liver structures.

The EPCs may also be used to help heal cardiac vasculature following angioplasty. For example, a catheter can be used to deliver embryonic endothelial cells to the surface of a blood vessel following angioplasty or before insertion of a stent. Alternatively, the stent may be seeded with embryonic endothelial cells. Blood vessels treated with adult endothelial cells exhibit accelerated re-endothelialization, preventing restenosis in the injured vessel (Parikh, et al. (2000) Advanced Drug Delivery Reviews, 42, 139-161). In another embodiment, embryonic endothelial cells may be seeded into a polymeric sheet and wrapped around the outside of a blood vessel that has undergone angioplasty or stent insertion (Nugent, et al. (2001) J. Surg. Res., 99, 228-234). The cells may also be mixed with a gel and infused into the polymer sheet instead of directly seeded onto the matrix.

If a stiffer implant is desired, the cells may be seeded onto a polymer matrix, for example, a sponge, which is then implanted into the desired tissue site. Alternatively, the cells may be mixed with a gel which is then absorbed onto the interior and exterior surfaces of the matrix and which may fill some of the pores of a spongy or other porous matrix. Capillary forces will retain the gel on the matrix before hardening, or the gel may be allowed to harden on the matrix to become more self-supporting. In certain embodiments, the polymer matrix is biodegradable. Suitable biodegradable matrices are well known in the art and include collagen-GAG, collagen, fibrin, PLA, PGA, and PLA-PGA co-polymers. Additional biodegradable materials include poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides. Non-biodegradable polymers may also be used as well. Other non-biodegradable, yet biocompatible polymers include polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, and poly(ethylene oxide). Those skilled in the art will recognize that this is an exemplary, not a comprehensive, list of polymers appropriate for tissue engineering applications.

In some embodiments, the matrix may be formed with a microstructure similar to that of the extracellular matrix that is being replaced. Mechanical forces imposed on the matrix by the surrounding tissue will influence the cells on the artificial matrix and promote the regeneration of extracellular matrix with the proper microstructure. The cross-link density of the matrix may also be regulated to control both the mechanical properties of the matrix and the degradation rate (for degradable scaffolds). The shape and size of the final implant should be adapted for the implant site and tissue type. The matrix may serve simply as a delivery vehicle for the cells or may provide a structural or mechanical function. The matrix may be formed in any shape, for example, as particles, a sponge, a tube, a sphere, a strand, a coiled strand, a capillary network, a film, a fiber, a mesh, or a sheet.

PLA, PGA and PLA/PGA copolymers are useful for forming the biodegradable matrices. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. PGA is the homopolymer of glycolic acid (hydroxyacetic acid). In the conversion of glycolic acid to poly(glycolic acid), glycolic acid is initially reacted with itself to form the cyclic ester glycolide, which in the presence of heat and a catalyst is converted to a high molecular weight linear-chain polymer. The erosion of the polyester matrix is related to the molecular weights. The higher molecular weights, weight average molecular weights of 90,000 or higher, result in polymer matrices which retain their structural integrity for longer periods of time; while lower molecular weights, weight average molecular weights of 30,000 or less, result in both slower release and shorter matrix lives. For example, poly(lactide-co-glycolide) (50:50) degrades in about six weeks following implantation.

In an exemplary embodiment, a cell response modifier such as a growth factor or a chemotactic agent may be added to the polymer matrix. Such a modifier, for example, vascular endothelial-derived growth factor (VEGF), may be used to promote differentiation of the EPCs. Alternatively, the modifier may be selected to recruit cells to the matrix or to promote or inhibit specific metabolic activities of cells recruited to the matrix. Exemplary growth factors include epidermal growth factor, bone morphogenetic protein, TGF-β hepatocyte growth factor, platelet-derived growth factor, TGF-α, IGF-I and II, hematopoetic growth factors, heparin binding growth factor, peptide growth factors, and basic and acidic fibroblast growth factors. In some embodiments it may be growth factors such as nerve growth factor (NGF) or muscle morphogenic factor (MMP). The particular growth factor employed should be appropriate to the desired cell activity. The regulatory effects of a large family of growth factors are well known to those skilled in the art.

The cell-seeded polymer matrix, with or without the gel, may be implanted into any tissue, including connective, muscle, nerve, and organ tissues. For example, an implant placed into a bony defect will attract cells from the surrounding bone which will synthesize extracellular matrix, while the EPCs form blood vessels. The blood supply for the new bone will be provided as the new ECM is formed and mineralized. An implant placed into a skin defect will promote dermis formation and provide a vascular network to supply nutrients to the newly formed skin.

Alternatively, the EPCs may be seeded onto a tubular substrate. For example, the polymer matrix may be formed into a tube or network. Such tubes may be formed of natural or synthetic ECM materials such as PLA or collagen or may come from natural sources, for example, decellularized tubular grafts. The EPCs will coat the inside of the tube, forming an artificial channel that can be used for a heart bypass. In addition, use of EPCs may reduce thrombosis post-implantation (see Kaushall, 2001). In certain embodiments, EPCs derived from iPS cells that are autologous to the patient can be used.

The EPCs may be allowed to proliferate on the polymer matrix or tubular substrate before being implanted in an animal. During proliferation, mechanical forces may be imposed on the implant to stimulate particular cell responses or to simulate the mechanical forces the implant will experience in the animal. For example, a medium may be circulated through a tubular substrate in a pulsatile manner (i.e., a hoop stress) or with sufficient speed to exert a sheer stress on cells coating the inside of the tube (Niklason, 1999; Kaushal, 2001). Alternatively, a hydrostatic force or compressive force may be imparted on an implant that will be deposited within an organ such as the liver, or a tensile stress may be imparted on an implant that will be used in a tissue that experiences tensile forces.

Cells that are recruited to the implant may also differentiate into other cell types. Bone cell precursors migrating into a bone implant can differentiate into osteoblasts. Mesenchymal stem cells migrating into a blood vessel can differentiate into muscle cells. Endothelial cells forming tubular networks in liver can induce the formation of liver tissue.

In another embodiment, the EPCs are mixed with another cell type before implantation. The cell mixture may be suspended in a carrier such as a culture medium or in a gel as described above. Alternatively, the cells may be co-seeded onto a polymer matrix or combined with a gel that is absorbed into the matrix. For some applications, it may be desirable to seed one cell type directly onto the matrix and add the second cell type via a gel. Any ratio of EPCs to the other cell type or types may be used. One skilled in the art will recognize that this ratio may be easily optimized for a particular application. Exemplary ratios of EPCs to other cells are at least 10% (e.g., 1:9), at least 25%, at least 50% (e.g., 1:1), at least 75%, and at least 90%. Smaller ratios, for example, less than 10%, may also be employed.

Any cell type, including connective tissue cells, nerve cells, muscle cells, organ cells, or other stem cells, may be combined with the EPCs. For example, osteoblasts may be combined with the embryonic endothelial cells to promote the co-production of bone and its vasculature in a large defect. Fibroblasts combined with embryonic endothelial cells and inserted into skin will produce fully vascularized dermis. Other exemplary cells that may be combined with the embryonic endothelial cells of the invention include ligament cells, lung cells, epithelial cells, smooth muscle cells, cardiac muscle cells, skeletal muscle cells, islet cells, nerve cells, hepatocytes, kidney cells, bladder cells, and bone-forming cells.

In certain embodiments, the EPCs of the present invention are used to target a therapeutic agent to a desired site in a subject. Desired sites include the vasculature at sites of neoplastic cell growth, e.g., tumor cells, in a subject. As well known in the art, tumors include regions of endothelial cell production, including both vascular and lymphatic endothelial cells. Tumor-associated vasculature has been shown to be important for tumor growth and maintenance. Tumor-associated lymphatic vessels have been shown to act as a conduit for disseminating tumor cells to form metastases, e.g., at lymph nodes, which is of major prognostic significance for many types of cancer. As noted above, EPCs according to aspects of the present invention are bipotent, i.e., can develop into either vascular or lymphatic endothelial cells. Therefore, EPCs according to aspects of the present invention find use as targeting vectors for delivering therapeutic agents to lymphatic and/or vascular endothelial cell sites in tumors. One example of employing EPCs as anti-tumor delivery vectors includes genetically modifying EPCs to express one or more anti-tumor (or cell toxic) protein or factor, including: cytokines, hormones or other signal transducing agents; antibody or antibody fragments, and the like. Another example of employing EPCs as anti-tumor delivery vectors includes conjugating or coating EPCs with anti-tumor factors or other toxic agents, including: antibodies or antibody fragments, cytokines, hormones, radioactive agents, cytotoxic agents, chemotherapeutic agents, and the like. No limitation in this regard is intended.

### Endothelial Tip Cells

In certain embodiments the invention provides methods of obtaining endothelial tip cells, e.g. by differentiating pluripotent stem cells in vitro into endothelial tip cells. Other embodiments of the invention provide in vitro cell cultures comprising endothelial tip cells. Still other embodiments of the invention provide for an isolated endothelial tip cell. The endothelial tip cells may be human endothelial tip cells.

Endothelial tip cells have the ability to give rise to new endothelial branches or tubules (del Toro et al. (2010) Blood 116:4025; Suchting et al. PNAS (2007) 104:3225). Endothelial tip cells express Dll4. Dll4 has the ability to inhibit new endothelial branches in neighboring endothelial vessels and thus acts as part of negative feedback loop to control new endothelial sprouting. Other markers found on endothelial tip cells include IGFBP-3, ESM-1, ang-2 and apelin.

The endothelial tip cells may be used in vitro to generate new endothelial vessels. The endothelial vessels may be used as therapeutics or as a research reagent to screen for drug effects and toxicity. The tip cells may also be used as a research tool to study endothelial vessel formation in vitro.

### Systems and Kits

Also provided by the subject invention are kits and systems for practicing the subject methods, as described above (generically referred to below as “kits”).

For example, kits may contain reagents and components for producing EPCs in vitro from pluripotent stem cells for either therapeutic or research purposes. In such embodiments, the kits may include such reagents as cytokines, cell culture media, enzymes (e.g., for cell dissociation, e.g., Accutase), antibodies and/or gene probes (e.g., antibodies or gene probes specific for LYVE-1, CD31, CD34, PV-1, etc.), culture plates or flasks, stocks of ES cells for use in the process, etc. Any reagent that finds use in producing and/or using the EPCs according to the present invention, or in performing quality control analyses on such EPCs, can be included.

In some embodiments, the kit is designed for endothelial cell production and includes one or more of the EPCs described herein and one or more additional components used for the propagation of the EPCs and/or for inducing endothelial cell production from the EPCs. Such systems and kits may be for therapeutic and/or research purposes.

The subject systems and kits may also include one or more other reagents for preparing or using EPCs according to the subject methods. The reagents may include one or more matrix or scaffold (or reagents for generating the matrix/scaffolds), hydrating agents (e.g., physiologically-compatible saline solutions, prepared cell culture media), cell culture substrates (e.g., culture dishes, plates, vials, etc.), cell culture media (whether in liquid or powdered form), antibiotic compounds, hormones, additives, etc. As such, the kits may include one or more containers such as vials or bottles, with each container containing a separate component for carrying out a processing or preparing step according to the present invention.

In certain embodiments, the kit may further include components designed to facilitate the delivery a cell population, e.g., to an experimental animal or to a patient in the need thereof, e.g., a patient in need of EPC-based therapy. In these latter embodiments, the components of the kit may be provided in a form that is suitable for therapeutic use (e.g., provided in as sterile/medical grade components). Delivery components can include those designed for encapsulating or immobilizing the cell population (e.g., a scaffold or matrix) as well as for delivering the cells, either directly or in association with other components (e.g., a scaffold or matrix), including injecting the isolated cells into the site of defect, incubating and/or culturing the embryonic progenitor cells with a suitable scaffold or matrix and implanting, incubating with bio-resorbable scaffold, etc. Any convenient scaffolds or matrices, such as bio-resorbable, bio-compatible scaffolds as described in detail above, may be employed, where a number have been employed for, or are being tested for use in, therapeutic endothelial repair, replacement or tumor targeting.

In some embodiments, the kit includes components for use in determining that the delivered/transplanted cell population locates to at least one desired site in a subject, e.g., a patient. Such components may allow the determination of the localization and even quantification of cells delivered cells to a subject.

In certain embodiments, the EPCs in the kit are genetically modified. For example, EPCs may be engineered to express an exogenous gene, e.g., a marker gene that can be used for later identification of cells derived from the EPCs (e.g., a reporter gene as is well known in the art). Reporter genes include those that are directly or indirectly detectable, e.g., fluorescent proteins, luminescent proteins, enzymes, cell surface markers, and the like. In certain embodiments, different cell lines re-engineered to express exogenous reporter genes that are discriminable from each other, e.g., fluorescent proteins having different excitation and/or emission characteristics.

In certain embodiments, the kit can include any or all components necessary for its intended use. For example, kits according to the invention may include a number of other suitable articles or components such as tubes, sutures, scalpels, needles, syringes, antiseptics for preparation of surgical sites, etc.

Additional types of kits are also provided in aspects of the present invention.

For example, kits are provided for the identification and/or isolation of EPCs according to the present invention. Such kits will include reagents designed for detecting the expression of cell markers including any of the gene expression markers described herein. Such detection reagents may be formulated to detect expression products of these genes at either at the protein or nucleic acid (e.g., mRNA) level. As such, reagents may include: antibodies or specific binding portions thereof (e.g., detectably labeled antibodies), other specific protein binding agents (e.g., ligands or soluble receptors), nucleic acid probes for use in hybridization analysis, e.g., northern blot analysis, microarray analysis, and the like; primer pairs for use in PCR assays, e.g., quantitative PCR assays, etc.

As noted above, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging), etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the components noted above, the kits may also include one or more control samples and reagents, e.g., two or more control samples. Such control samples may take any form, e.g., additional cell lines having known marker profiles, negative and positive control samples for use in analyzing gene expresison data, etc. Any convenient control sample may be employed in the subject kits.

In further embodiments the invention provides a system for making endothelial cells comprising a first population of cells comprising pluripotent stem cells, such as embryonic stem cells and a second population of cells comprising endothelial progenitor cells. In yet other embodiments the invention provides a system for making endothelial cells comprising a first population of cells expressing the markers TERT, OCT4, SSEA4 and TRA-160 and a second population of cells comprising endothelial progenitor cells. In some embodiments the first and second populations described above are in the same container. In other embodiments each of the cell populations are contained in separate containers.

Because pluripotent stem cells, such as established embryonic stem cell lines, have the ability to be maintained in culture as pluripotent cells for prolonged periods of time, the invention provides a system for producing virtually unlimited amounts of in vitro differentiated cell populations, such as endothelial progenitors. That is stem cells can replicate in culture as pluripotent cells and those cells can be used according to the methods described infra to make endothelial progenitor cells and the endothelial progenitor cells can proliferate in culture and can be used to make endothelial cells, such as vascular endothelial cells and lymphatic endothelial cells. Moreover, because the stem cell and their differentiated progeny share a genome that is essentially the same, the system eliminates genetic variability to a large extent in producing differentiated somatic cells of any type, including endothelial progenitor cells, such as bipotential progenitor cells having the ability to differentiate into both vascular endothelial cells and lymphatic endothelial cells. This provides for a means of producing, in a reproducible manner, an unlimited supply of endothelial progenitor cells having essentially the same genome. Thus the parental stem cell line and the differentiated endothelial progenitor cells (i.e. the various batch runs of endothelial progenitor cells produced from the same starting material, e.g. the stem cell line) all have essentially the same genome. This provides a for reproducibility in developing products for research (e.g. drug discovery and toxicity screening) and therapy (for treating conditions such as heart disease and cancer).

Cells (i.e. the parental stem cell line and the differentiated progeny, e.g., endothelial progenitor cells derived from the parental stem cell line) having essentially the same genome may be about 96%, about 97%, about 98%, about 99%, about 99.9% genetically identical. Cells (i.e. the parental stem cell line and the differentiated progeny endothelial progenitor cells derived from the parental stem cell line) having essentially the same genome may be at least 96%, at least 97% at least 98%, at least 99%, at least 99.9% genetically identical. Cells (i.e. the parental stem cell line and the differentiated progeny, e.g., endothelial progenitor cells derived from the parental stem cell line) having essentially the same genome may have variability in about 1%, about 2%, about 3% about 4%, about 5% of their genomes. Genetic identity may be determined using any method known in the art. For example the genome of both parental stem cell line and the differentiated progeny may be sequenced. Alternatively the genomes of both the parental cell line and the differentiated progeny may be analyzed using restriction enzyme analysis.

### Combinations

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to EPCs are specifically embraced by the present invention and are disclosed herein just as if each and every combination was individually and explicitly disclosed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Production of Human Endothelial Progenitor Cell (hEPC) from ES Cells

Human Embryonic Stem Cells (hESCs)

hESCs of various lines (e.g., H1, H9, ESI 017, ESI 035, ESI 051) were routinely maintained in mTeSR1™ medium (Stem Cell Technologies, Vancouver, BC) on Growth Factor-Reduced Matrigel-coated T flasks or cell culture plates. The cells were detached and harvested for experiments using Accutase when colonies became 50-80% confluent.

### Embryoid Bodies (EBs)

To produce EBs of uniform size, 2.4 to 3.0 million ES cells in complete Stemline II basal medium (containing 1× Glutamax, 1× penicillin/streptomycin, and 50 μM β-mercaptoethanol) and also containing 10 uM Y27632 Rock inhibitor were added to each well of AggreWell 400 plates (Stem Cell Technologies; ca. 1200 microwells/well in a 24 well plate format). The plates were centrifuged essentially according to the manufacturer's supplied directions, depositing 2000 to 2500 cells/microwell, and the plates were then cultured in a 5% O₂, 5% CO₂, 37° C. incubator. The following day (at about 24 hours), recombinant human BMP4 (human cell-expressed, HumanZyme, Chicago, Ill.) was added to 20 ng/ml. In some experiments, recombinant human Activin A (HumanZyme) was added to 10 ng/ml at the same time. Alternatively, the Activin A was added the next day (about 48 hours). FIG. 3 shows an EB formation in AggreWell™ 400 plates in exemplary protocols with 4 different concentrations of cells (cells/μwell). FIG. 4 shows EBs harvested from AggreWell™ 400 and 800 plates (2000 cells/μwell and 8000 cells/μwell, respectively) at 24 hours as compared to EBs formed free in culture.

On day 2 (after about 48 hours) the EBs were collected and transferred to 6 well Costar 3471 ultra low attachment (ULA) plate (1 AggreWell plate to 1 ULA plate), in complete Stemline II basal medium containing 20 ng/ml BMP4, 10 ng/ml Activin A, and 8 ng/ml of FGF2 (recombinant human FGF-basic, Gibco PHG0263), and incubation continued at 5% O₂, 10% CO₂, 37° C.

hEPC Differentiation and Expansion

At day 5 the EBs were collected and dissociated with Accutase. The cells were then passaged to fibronectin-coated (e.g., human plasma-derived from, Becton Dickinson or bovine plasma-derived from Sigma) T flasks in complete Stemline II basal medium containing 10 ng/ml BMP4, 8 ng/ml FGF2, 25 ng/ml VEGF-165 and 10 μM Y27632 (“Adherent-1 medium”) and culture was continued in a 5% O₂, 10% CO₂, 37° C. incubator.

At day 8 the adherent cells were harvested using Accutase and cultures were expanded thereafter by passaging to tissue culture treated T flasks in complete Stemline II basal medium containing 8 ng/ml FGF2, 25 ng/ml VEGF-165, and 10 μm TGFβ signaling inhibitor SB431542 (“Adherent-2 medium”) and maintained in an ambient O₂, 5% CO₂, 37° C. incubator.

As cultures approached confluence, they were split and passaged further. Between days 14-20 the cultures were harvested using Accutase. Optionally, the cells were passaged further, used for assays, cryopreserved (using serum-free medium such as Cryo-SFM (Promocell) or 5-10% DMSO/40-90% FBS) and stored at liquid nitrogen temperature. Optionally also, they were subjected to isolation (i.e., enrichment or purification by positive or negative cell selection) of subpopulations expressing specific membrane antigens such as CD31 or CD34 by FACS or by immunomagnetic selection using, e.g., CD31 or CD34 Microbead kits (Miltenyi Biotec) Dynal CD34 Progenitor Cell Selection System (Invitrogen) for research purposes, the clinical-grade versions of these kits and devices, or antibodies or ligands to other antigens or molecules expressed by the cells along with magnetic beads or other matrices. These options were used in any combinations appropriate to the purposes of experiments.

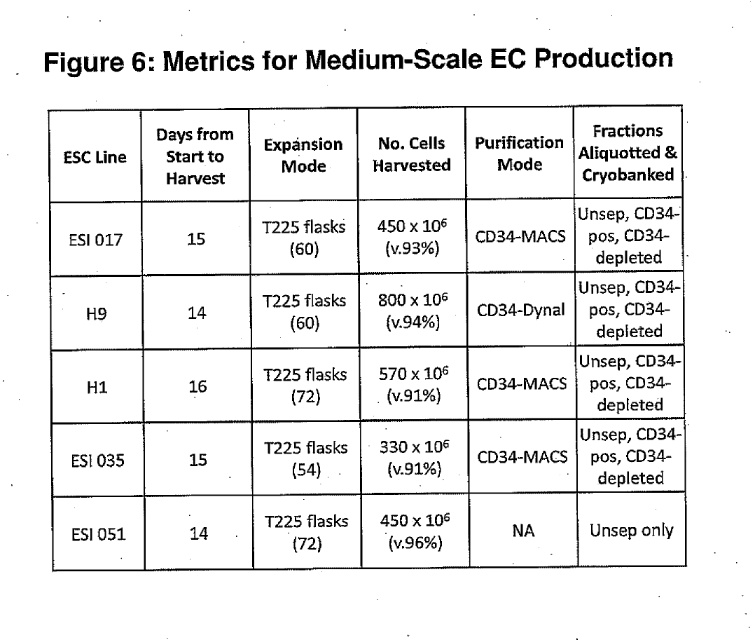

FIG. 6 shows metrics for several exemplary medium-scale EPC production processes performed according to aspects of the present invention. The table shows the days from start of the culture to harvesting the EPCs, the expansion mode (flask type and number used), the number of cells harvested and the % viability, the purification mode used, and the fractions collected for further analyses and processing.

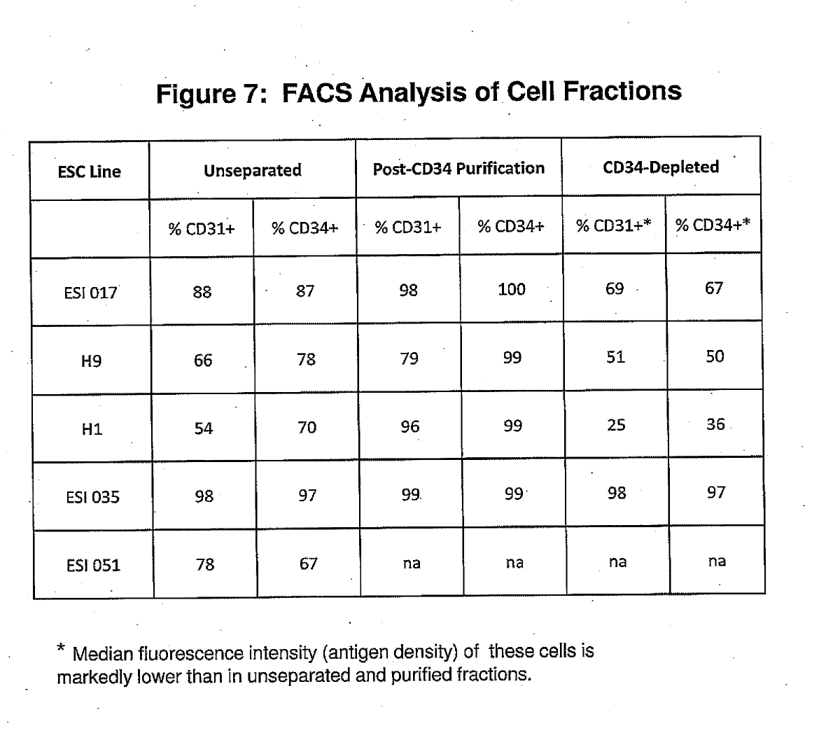

FIG. 7 shows FACS Analysis of cell fractions as described in FIG. 6. Percentages of positive cells for CD31 and CD34 are shown for each of the cell lines listed before separation (unseparated), or after CD34 enrichment (purification), or after CD34 depletion.

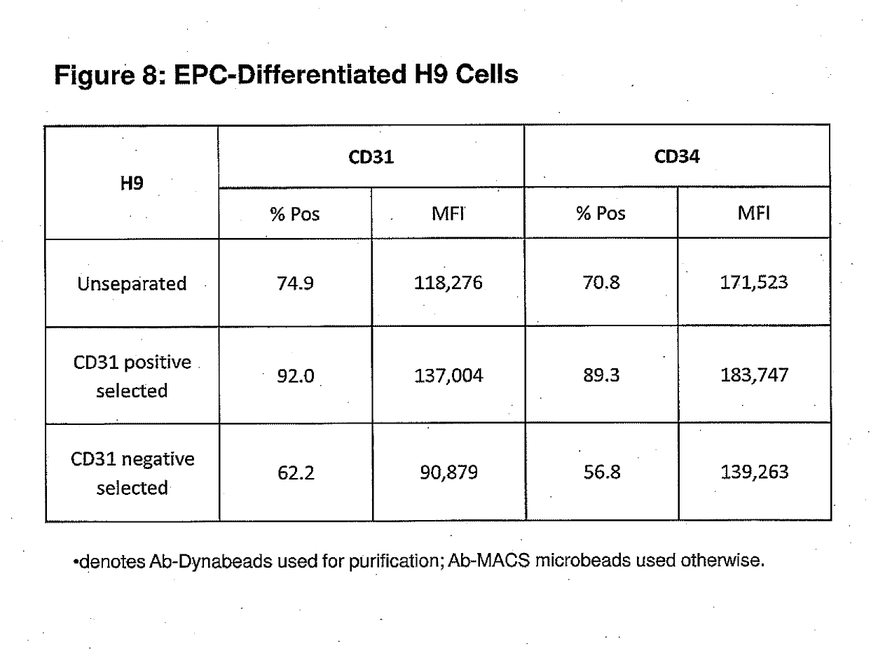

FIG. 8 shows the analysis of different fractions of EPC-differentiated H9 Cells. Percent positive cells in the specified populations and mean fluorescence intensity (MFI) are shown for CD31 and CD34 cell markers. Differentiated H9 cells were analyzed as unseparated cells, positively selected for CD31, or negatively selected for CD31.

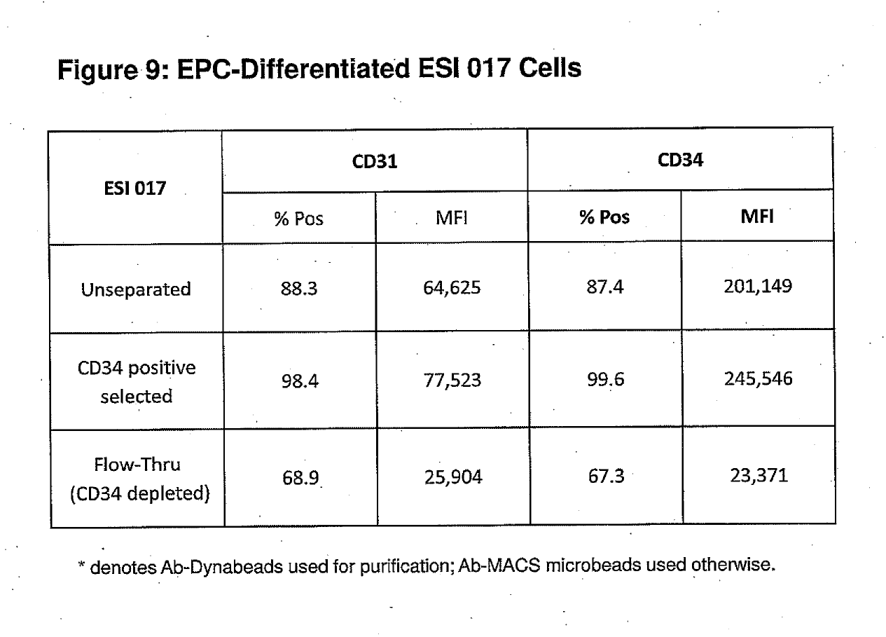

FIG. 9 shows analysis of different fractions of EPC-differentiated ESI 017 Cells. Percent positive cells in the specified populations and mean fluorescence intensity (MFI) are shown for CD31 and CD34 cell markers. EPC-differentiated ESI 017 cells were analyzed as unseparated cells, positively selected for CD34, or CD34 depleted.

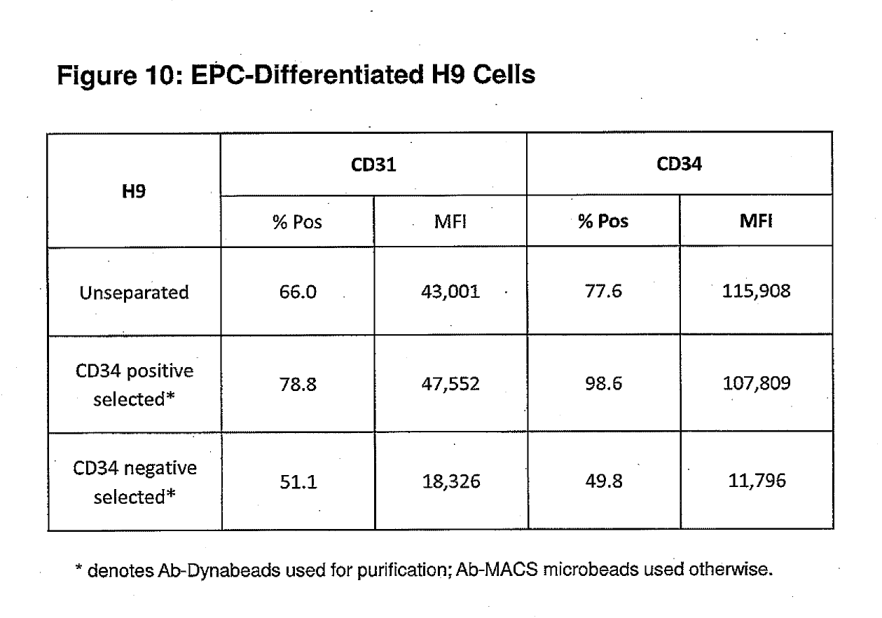

FIG. 10 shows analysis of different fractions of EPC-differentiated H9 Cells. Percent positive cells in the specified populations and mean fluorescence intensity (MFI) are shown for CD31 and CD34 cell markers. EPC-differentiated H9 cells were analyzed as unseparated cells, positively selected for CD34, or CD34 depleted.

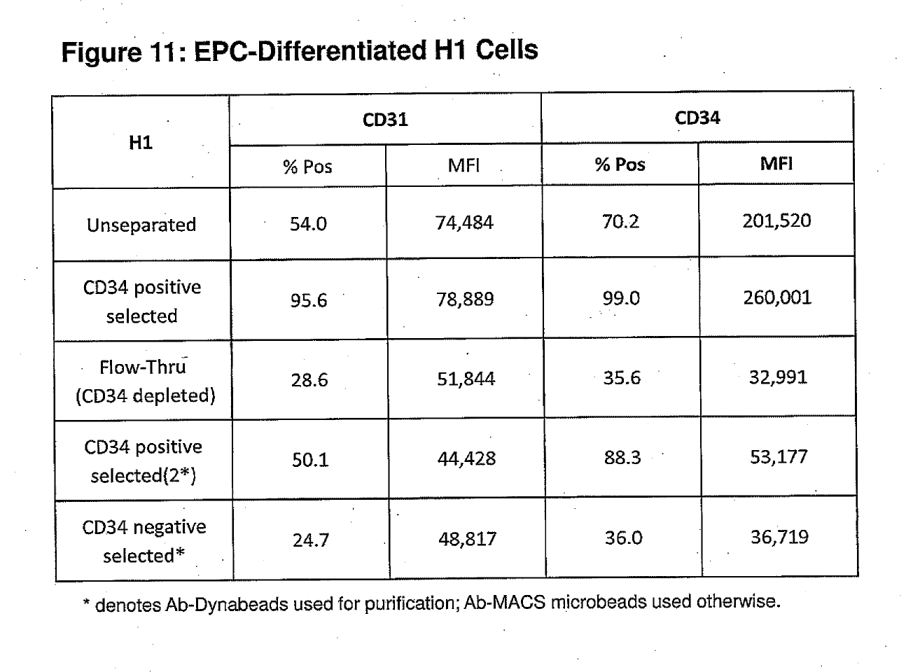

FIG. 11 shows analysis of different fractions of EPC-differentiated H1 Cells. Percent positive cells in the specified populations and mean fluorescence intensity (MFI) are shown for CD31 and CD34 cell markers. EPC-differentiated H1 cells were analyzed as unseparated cells, positively selected for CD34 (using either Dynabeads or MACS beads), or CD34 depleted (using either Dynabeads or MACS beads).

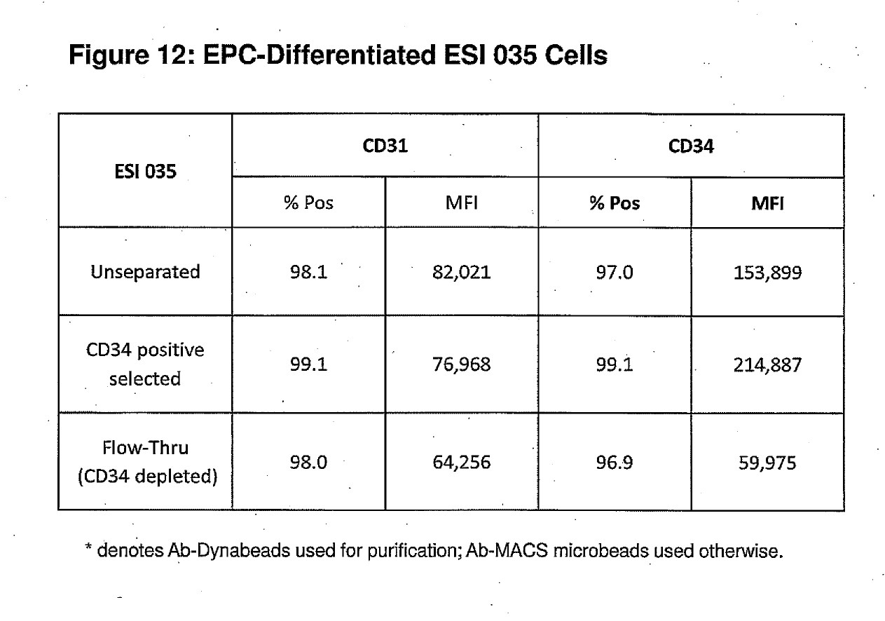

FIG. 12 shows analysis of different fractions of EPC-differentiated ESI 035 Cells. Percent positive cells in the specified populations and mean fluorescence intensity (MFI) are shown for CD31 and CD34 cell markers. EPC-differentiated ESI 035 cells were analyzed as unseparated cells, positively selected for CD34, or CD34 depleted.

It is noted that BMP4, Activin A and VEGF-165 employed in the exemplary EPC differentiation and expansion protocol above are recombinant Human factors expressed in a Human cell line (from HumanZyme). Thus, these factors have Human glycosylation and may have higher potency and/or stability in culture than the same recomobinant Human factors expressed in E. coli (e.g., from Peprotech) or in mouse NS0 (e.g., from R&D Systems), and thus could be used at lower concentrations than recited above.

### Example 2

### Gene Expression Analysis of EPCs

### A. Expression of LYVE-1 in EPCs

Day 15 cultures of H9 (WA-09) ES cell-derived EPCs were positively selected or depleted for CD31 antigen expression using CD31 MicroBead Kits (Miltenyi Biotec) according to the kit instructions. The positively selected cells were cultured in T flasks for another 6 days directly on the tissue culture treated plastic surface (M+SP), on a layer of Matrigel (M+SM, or in complete EGM-2 medium (M+EGM). The flow-through fraction from the CD31 MicroBead selection (CD31 depleted but containing residual CD31 positive cells) was secondarily sorted into CD31 positive (D+SP) and negative (more highly depleted); (D−SP) fractions using a Dynabeads CD31 kit (Invitrogen), and these cells were also cultured for another 6 days on tissue culture treated plastic. The cultures were then harvested and RNA was extracted for analysis.

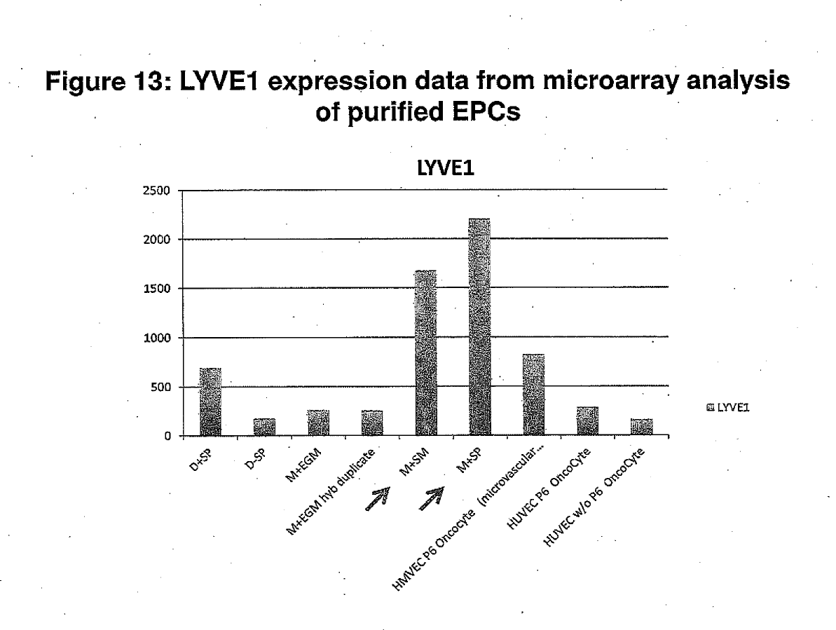

FIG. 13 shows LYVE-1 expression data from microarray analysis of the purified EPCs showing a positive correlation between the amount of CD31 antigen expression and LYVE-1 RNA expression. Human microvascular endothelial cells (HMVEC, passage 6) and human umbilical vascular endothelial cells (HMVEC) were used as reference controls.

This date demonstrates shows the EPCs produces according to aspects of the present invention have lymphatic vascular differentiation potential.

B. Analysis of Gene Expression in Cryobanked EPC from Different hESCS

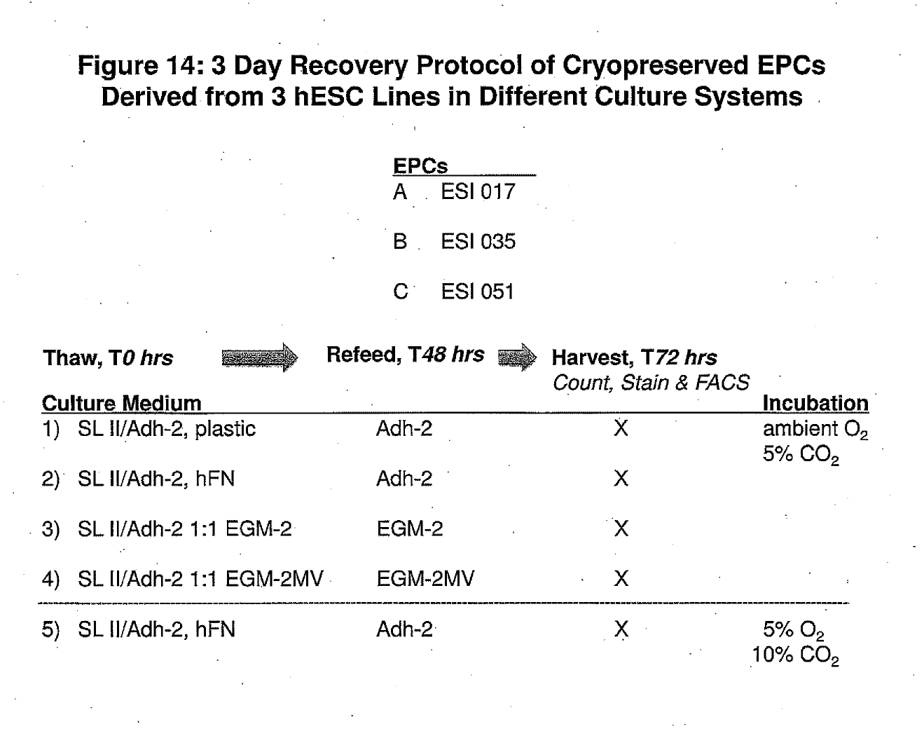

FIG. 14 shows different 3 day recovery protocols for culturing cryopreserved EPCs derived from 3 hESC lines (listed at top). In this study, the cryopreserved EPCs were derived from the 3 ESI ES cell lines indicated using the 15 day protocol with Stemline II basal medium and then cryopreserved and stored in liquid nitrogen. For the purposes of this experiment, unpurified (i.e., not immune-selected) cells were then thawed and returned to culture at 37° C. in T flasks in Stemline II medium containing aherent phase 2 factors (FGF-2, VEGF-165, SB431542) directly on the flask plastic surface (condition 1); in the same medium but on human fibronectin coated (condition 2); in a 1:1 mixture of Stemline II with adherent 2 factors and EGM-2 medium, then re-fed at 48 hours with EGM-2 medium only (condition 3); in a 1:1 mixture of Stemline II with adherent 2 factors and EGM-2MV medium, then re-fed at 48 hours with EGM-2MV medium only (condition 4); in the same medium on fibronectin as condition 2, but at 5% O₂, 10% CO₂ incubation (condition 5) rather than ambient O₂ as in the previous conditions

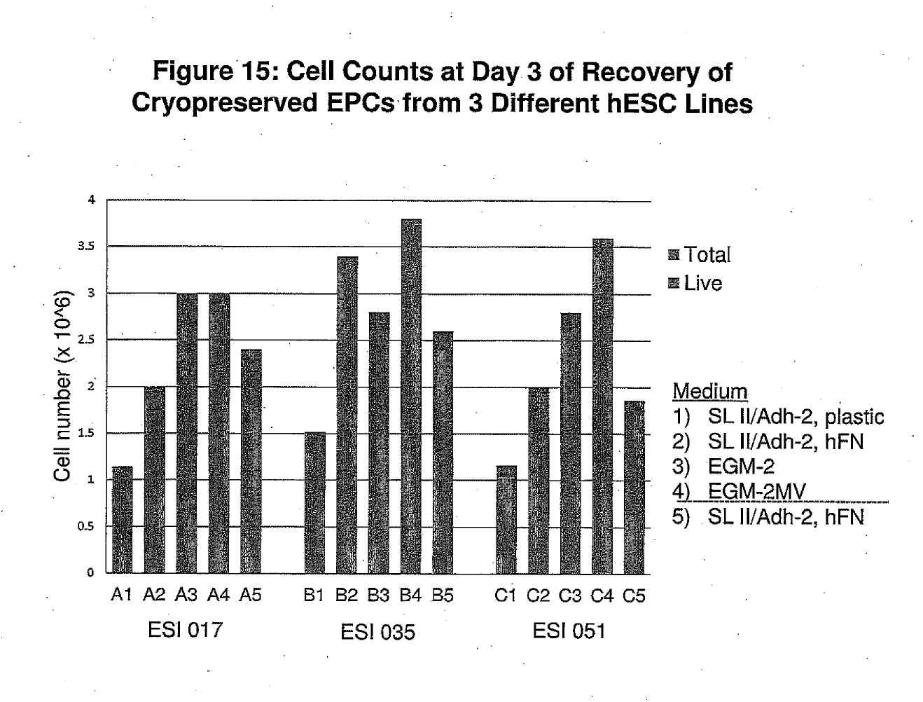

FIG. 15 shows cell counts for 3 day recovery protocols of FIG. 14. Total cell recovery and live cell recovery are shown. Each different culture condition for each different line are given an alpha-numeric designation used to identify the cells in subsequent Figures (shown on the x-axis).

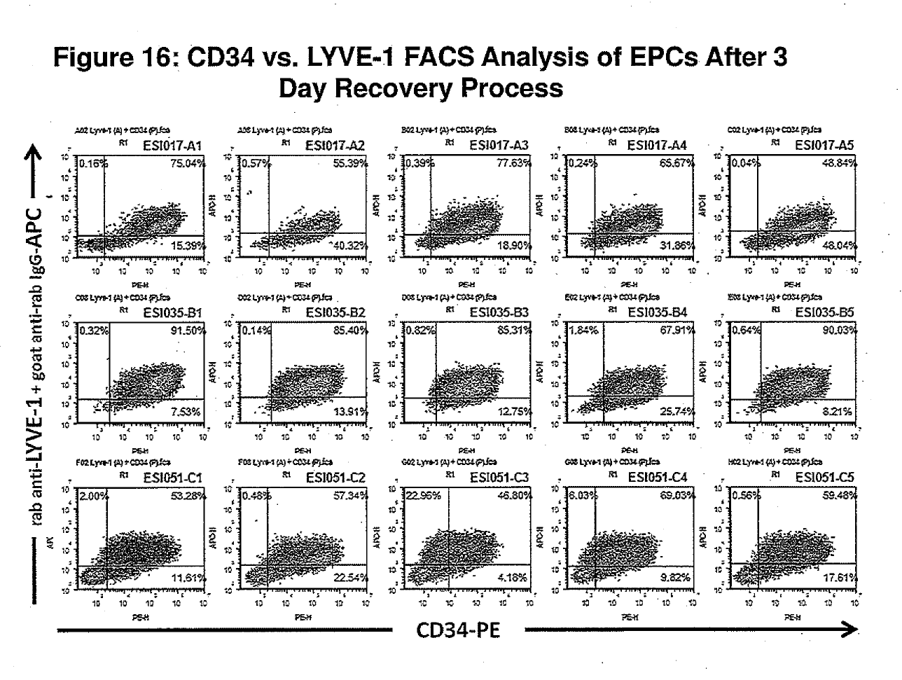

FIG. 16 shows FACS plots of CD34 vs. LYVE-1 expression on cells in FIG. 15. Gating was based on non-specific isotpye-control staining (not shown). Significant numbers of cells are positive for both markers in each of the cultures tested.

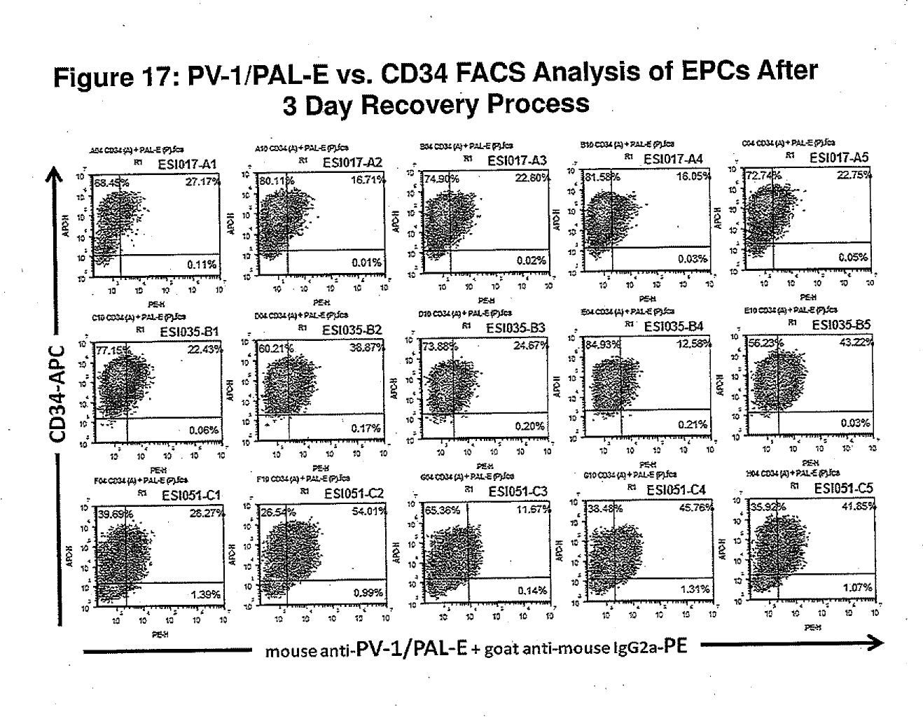

FIG. 17 shows FACS plots of PV-1/PAL-E vs. CD34 FACS expression on cells in FIG. 15.

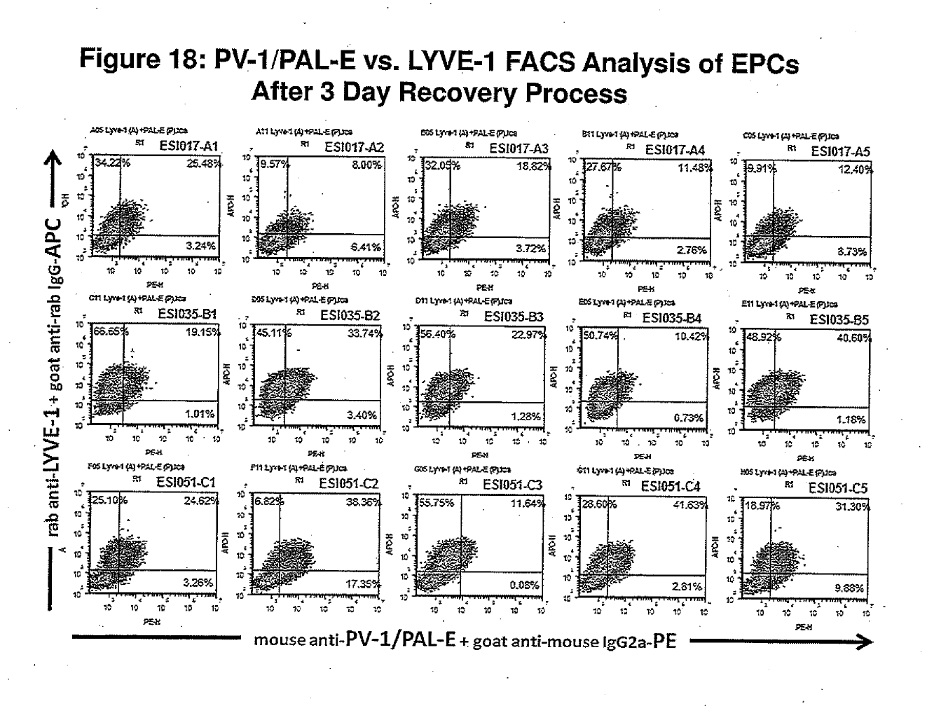

FIG. 18 shows FACS plots of PV-1/PAL-E vs. LYVE-1 FACS expression on cells in FIG. 15. LYVE-1 is co-expressed on ca. 51-94% of CD34+ cells. PAL-E is co-expressed on ca. 8-37% of CD34+ cells. LYVE-1 and PAL-E are co-expressed on ca. 8-46% of cells. Percentages can be shifted within certain ranges by varying the culture conditions (Stemline +/−FN, ambient O₂ vs. hypoxia, EGM-2, or EGM-2MV) for 72 hours. The range is very characteristic for each ESI line. LYVE-1 is known to be a lymphatic endothelial cell marker while PAL-E (PV-1) is a marker of vascular endothelial cells. Co-expression of these previously exclusive markers in EPCs as detailed herein indicates that these EPCs are bipotent for both lymphatic and vascular endothelial cells.

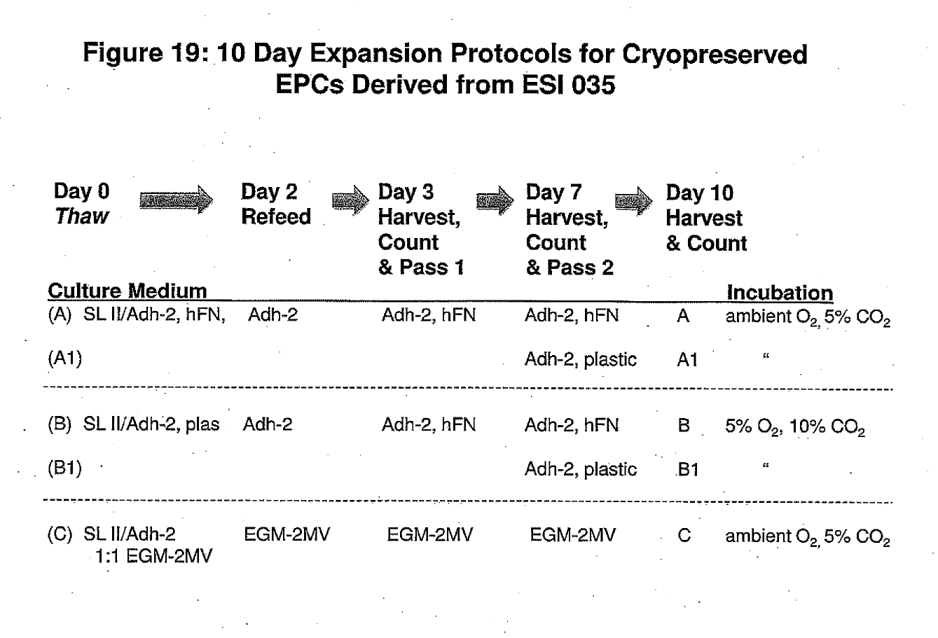

FIG. 19 shows different 10 Day expansion protocols for culturing cryopreserved EPCs derived from ESI 035.

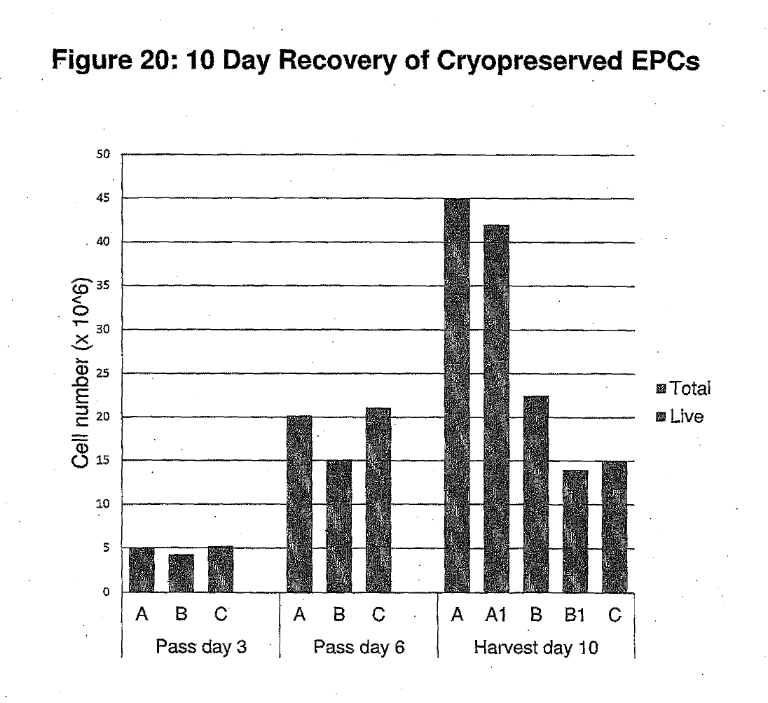

FIG. 20 shows cell counts for different steps of the 10 day expansion protocols of FIG. 19.

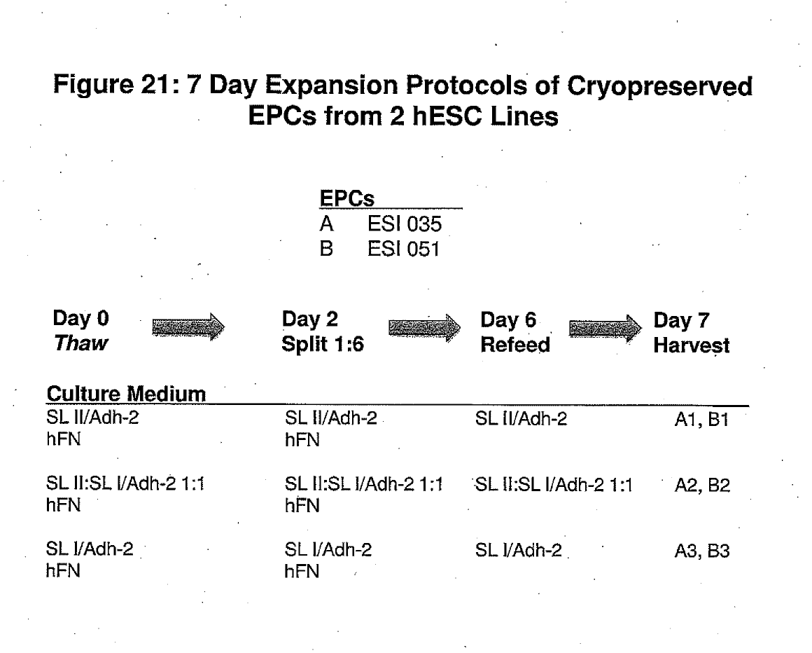

FIG. 21 shows different protocols for 7 day expansion of cryopreserved EPCs from 2 hESC lines (listed at top).

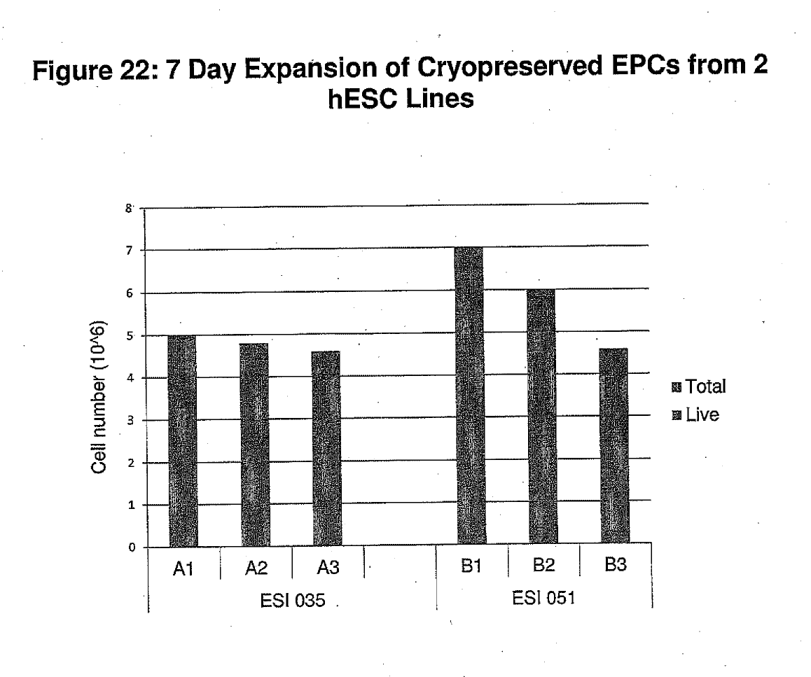

FIG. 22 shows cell counts for 7 day expansion protocols of FIG. 21.

### FACS Methods

| Comparison | Primary Antibodies | Secondary Antibodies |
|---|---|---|
| LYVE-1 vs. | Rabbit anti-LYVE-1 (Acris) | Goat anti-rabbit IgG- |
| CD34 | CD34-PE (IgG1; Biolegend) | APC (Leinco) |
| PV-1(PAL-E) | Mouse anti-PV-1/PAL-E | Goat anti-mouse IgG2a- |
| vs. CD34 | (IgG2a; Thermo) | PE (SoBio) |
| | CD34-APC (IgG1; Biolegend) | |
| LYVE-1 vs. | Mouse anti-PV-1/PAL-E | Goat anti-mouse IgG2a- |
| PV-1(PAL-E) | (Thermo) | PE (SoBio) |
| | Rabbit anti-LYVE-1 | Goat anti-rabbit IgG- |
| | (Acris Antibodies) | APC (Leinco) |

Key:
APC, allophycocyanin conjugate;
PE, phycoerythrin conjugate;
Acris (Acris Antibodies, San Diego, CA);
Biolegend (Biolegend, San Diego, CA);
Leinco (Leinco Technologies, St. Louis, MO);
SoBio (Southern Biotech, Birmingham, AL);
Thermo (Pierce/Thermo Scientific, Rockford, IL)

Cells were immunostained for (co)expression of different cell surface antigens using fluorochrome conjugates of both primary (direct) and secondary (indirect) antibodies as appropriate for the staining combinations. Negative staining controls were matched for Ig isotype, species and source. Single and multi-color FACS data acquisition was performed using an Accuri C6 Flow Cytometer (Ann Arbor, Mich.), and data analysis was performed using Accuri C Flow Plus or FCS Express (DeNovo Software, Los Angeles, Calif.).

### Example 3

Most solid tumors eventually require the formation of neo-vasculature for continued growth and metastases. This dependence on angiogenesis has been exploited in anti-cancer therapies with monoclonal antibodies, small molecule inhibitors, and cell-based approaches. Among the latter strategies is a so-called “Trojan horse” approach that includes ex vivo derivation and “arming” of tumor-homing cells followed by their systemic delivery and release of a toxic payload at the tumor.

As a first step toward such a Trojan horse approach, we have established a highly efficient process for the derivation of endothelial progenitor cells (EPCs) from human embryonic stem cells (hESCs). Our process to date has reproducibly provided high cell yields (>4×10⁸ cells) and purities (approaching 99%) within 2-3 weeks of culture initiation. These cells display phenotypic (morphology, cell surface antigen, and gene expression) and functional vascular endothelial cell characteristics (tube formation in vitro and incorporation into both normal and tumor neo-vasculature in vivo). Our derivation process has been applied to 5 independent hESC lines, including H1 (WA01) and 149 (WA09), as well as 3 of our GMP-compliant hESC lines (ESI 017, ESI 035 and ESI 051). The process uses a chemically-defined medium with sequential additions of recombinant human cytokines and signaling inhibitors, incorporates an optional immunomagnetic sorting step for CD31 or other characteristic markers, and is scalable to clinical demands. It does not involve feeder cells, serum, or xenogeneic components. Furthermore, our EPCs can be cryopreserved and banked, and are recovered with high efficiencies and the preservation of endothelial phenotypic characteristics.

In vitro-derived or expanded endothelial progenitor cells (EPCs) have clinical applications for targeting sites of tumor neovascularization in order to deliver cytotoxic or cytostatic payloads. They are also useful for targeting sites of vascular injury in order to initiate or accelerate tissue repair/regenerative processes.

Methods

The EPC methods described below were done as schematized in FIGS. 1 and 2; note that the day on which steps were performed recited below are according to FIG. 1, which is +1 day with respect to FIG. 2 (noted above).

ESC Maintenance.

NIH-registered hESC lines WA01 (H1) and WA09 (H9), along with hESC lines ESI-017 (NIH-registered), ESI-035 and ESI-051 (pending NIH registration) were maintained and expanded in growth factor-reduced Matrigel (BD Biosciences)-coated T150 or T175 flasks in mTeSR™I medium (Stem Cell Technologies). The ESI lines were research grade versions of cGMP-compliant lines made by Embryonic Stem Cells International, a BioTime subsidiary; see Crook et al. (2007) The Generation of Six Clinical-Grade Human Embryonic Stem Cell Lines. (see Cell Stem Cell 1:490-494, and www(dot)biotimeinc(dot)com).

Embryoid Bodies (EBs).

EBs of optimal and uniform size for EPC production (data not shown) were generated by forced aggregation in AggreWell 400 plates (Stem Cell Technologies). After 2 days the EBs were harvested and transferred to 6-well ultra-low attachment plates (Corning) and incubated for an additional 3 days at 5% O₂, 10% CO₂, 37° C. Recombinant human cytokines BMP4, Activin A (HumanZyme) and FGF2 (Invitrogen) were added according to the flow scheme depicted in FIG. 2, modified from James et al. (2010) Nature. Following preliminary studies comparing different basal culture media for EB formation and downstream derivations (data not shown), we chose for the majority of work shown here the chemically-defined medium Stemline II (Sigma), which has an FDA-registered Drug Master File.

Endothelial Adherent Culture Stage 1.

On day 5 the EBs were harvested and dissociated with Accutase, then transferred to T150 or T225 flasks coated with human fibronectin in Stemline II medium containing BMP4, FGF2, VEGF-165 (HumanZyme) and Y27632 rock inhibitor, and incubated for 3 more days at 5% O₂, 10% CO₂, 37° C.

Endothelial Adherent Culture Stage 2.

On day 8 the cells were harvested using Accutase and expanded thereafter (typically split 1:4.5-6) in uncoated T225 flasks in Stemline II medium containing FGF2, VEGF-165, and TGF-β signaling inhibitor SB431542 (Sigma or Talecris; James et al., ibid) at ambient O₂, 5% CO₂, 37° C. incubation. Between days 14-16 the cultures were typically harvested for assays (FACS, OCR, RNA microarrays), CD31- or CD34-positive cell immunomagnetic selections (Miltenyi Biotec Microbead or Invitrogen Dynabead kits), expanded further, and/or cryopreserved (Cryo-SFM (PromoCell) or DMSO/FBS) and stored at LN₂ temperature. Thawed cells were returned to culture under similar conditions.

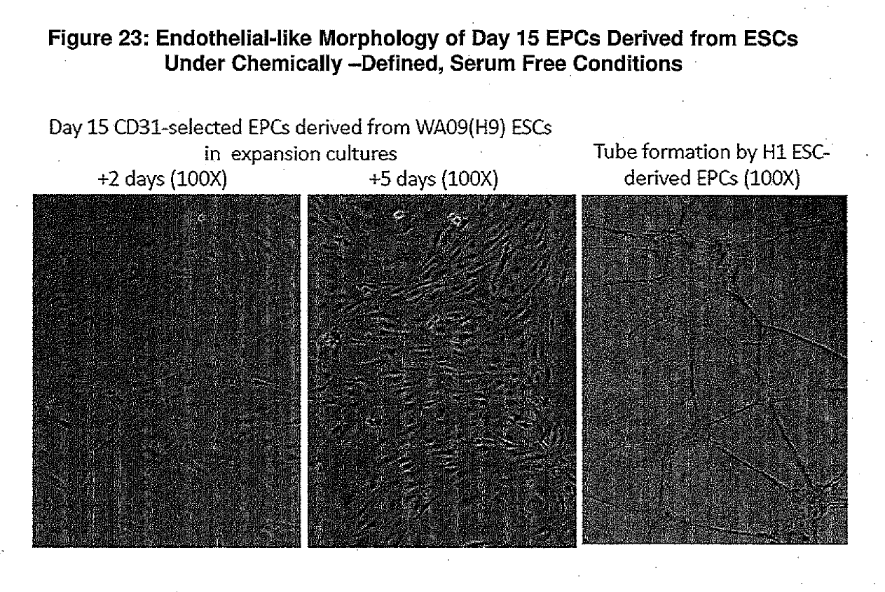

FIG. 23 shows the endothelial-like morphology of day 15 EPCs derived from ESCs as described above.

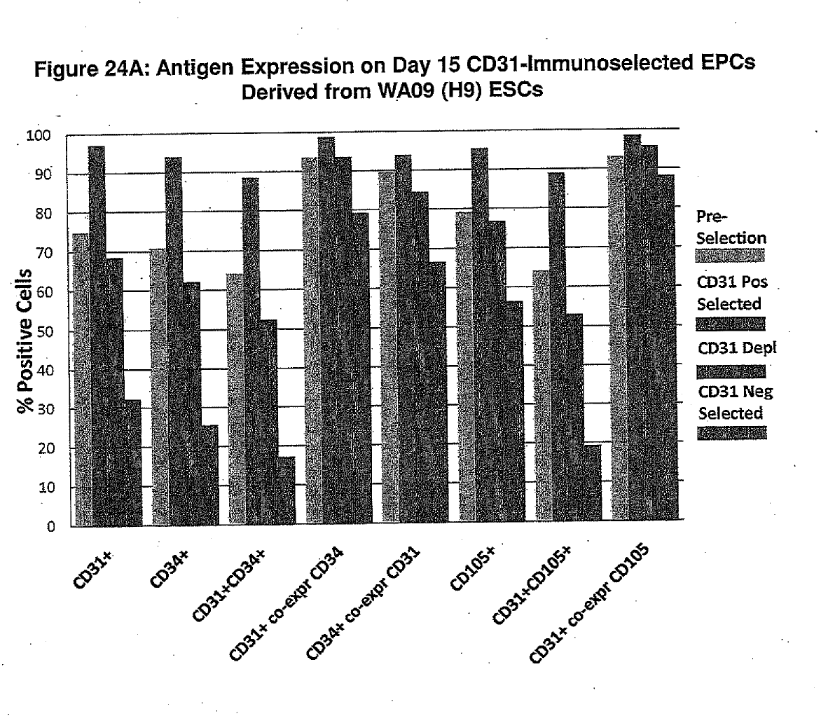

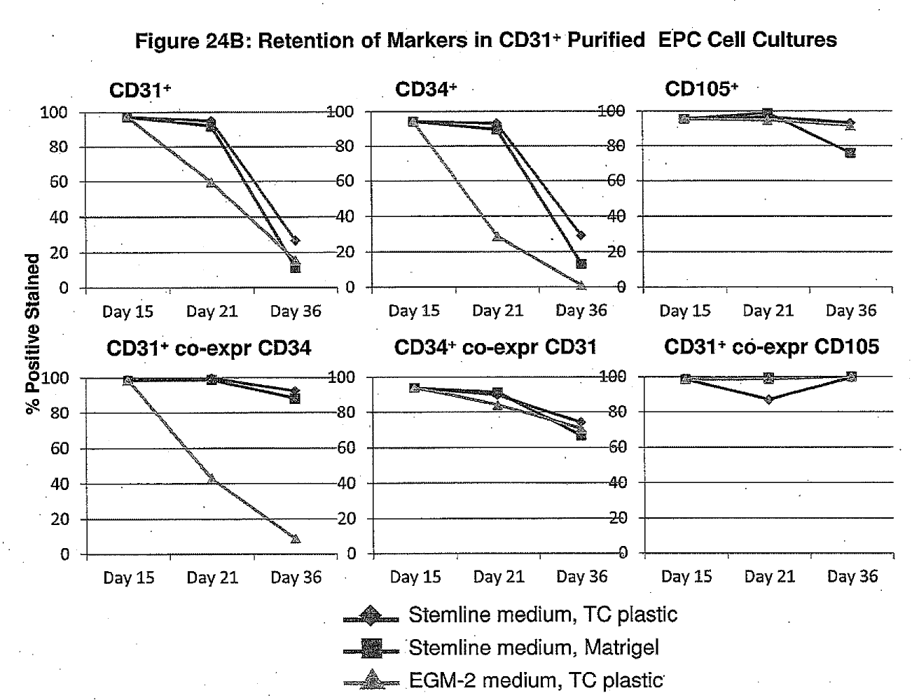

FIG. 24 shows antigen expression of day 15 CD31-immunoselected EPCs derived from WA09 (H9) ESCs. Cells harvested from day 15 EPC differentiation cultures started from H9 ESCs (“Pre-Selection”) were purified (“CD31 Pos Selected”) using CD31-MACS Microbead kits (Miltenyi Biotec). Residual CD31⁺ cells in the column flow-through fraction (“CD31 Depleted”) were depleted further (“CD31 Neg Selected”) using CD31-Dynabead kits (Dynal/Invitrogen). All cell fractions were immunostained for the antigens indicated using direct MAb PE and APC conjugates from multiple sources (with similar results) and analyzed using an Accuri C6 flow cytometer. Fluorescence gating was performed for each cell fraction using controls matched to the maximum extent possible for source, Ig isotype and conjugate. Among the conclusions reached from these and related analyses (data not shown) was that CD34 was at least as reliable a marker for EPCs as CD31 at this stage in our process, and a more useful antigen for clinical cell selections. FIG. 24B shows a time course of antigen expression on day 15, day 21, and day 36 CD31-immunoselected EPCs derived from WA09 (H9) ESCs.

FIG. 6 (as described in the previous Examples) shows a summary of general parameters for 5 consecutive EPC production runs from different ESC lines. Immunoselections were performed for CD34⁺ cells using CD34-MACS Microbead kits or CD34-Dynabead (DETACHaBEAD) kits (where indicated in the table). These studies demonstrate the consistency of the process.

FIG. 7 (as described in the previous Examples) shows a summary of CD31 and CD34 antigen expression in the different cell fractions of the EPC production runs described in FIG. 6. All cell fractions were immunostained for the antigens indicated (as well as other markers not shown) using direct MAb PE and APC conjugates from multiple sources (with similar results) and analyzed using an Accuri C6 flow cytometer. Fluorescence gating was performed for each cell fraction using controls matched to the maximum extent possible for source, Ig isotype and fluorochrome conjugate. Among the conclusions reached from these and related analyses (data not shown) were that (a) CD34⁺ cell purification results in substantial co-enrichment of CD31⁺ cells, (b) these two antigens are more similarly co-expressed in EPCs derived from the ESI ESC lines than from H1 and H9 lines, and (c) residual CD34 antigen-positive cells occur in the “Depleted” fractions.

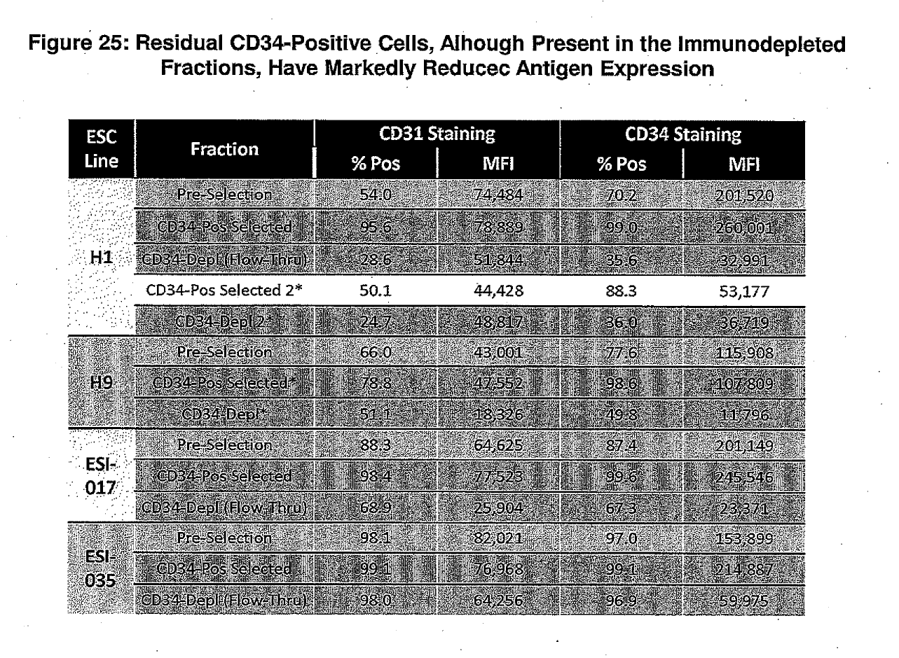

FIG. 25 shows further analyses of cell fractions from the preceding tables showing: (1) consistently greater mean fluorescence intensities (MFIs) for CD34 expression than for CD31 on Unselected and CD34-Positive Selected cells; and (2) 8-10-fold differences for CD34 compared to 1.5-3-fold differences for CD31 in MFIs of the CD34-Positive Selected vs. CD34-Depleted fractions (except for ESI-035-derived EPCs which had almost no CD31- or CD34-negative cells). MAbs from different sources and hybridoma clones and with the reverse PE and APC conjugations gave similar staining results.

As described above, FIGS. 16-18 show cryopreserved EPCs (unselected) from each of the production runs described above re-established in culture under 5 different conditions to determine if this could influence their growth, further differentiation and phenotype over time.

Upper rows in FIGS. 16-18 are ESI-017-derived EPCs;

Middle rows are ESI-035-derived EPCs;

Lower rows are ESI-051-derived EPCs.

The 5 culture conditions corresponding to columns 1-5 in FIGS. 16-18 are, respectively:

(1) Stemline medium with stage 2 factors, TC plastic-treated flasks, ambient O₂, 5% CO₂ incubation;

(2) similar to condition 1 but on fibronectin-coated flasks;

(3) 1:1 mixture of Stemline medium with factors and EGM-2 medium (Lonza), weaned at 48 hrs into EGM-2 alone at ambient O₂, 5% CO₂ incubation;

(4) similar to condition 3 but with EGM-2MV (microvascular) medium (Lonza) instead of EGM-2; (5) similar to condition 1, but at 5% O₂, 10% CO₂ incubation.

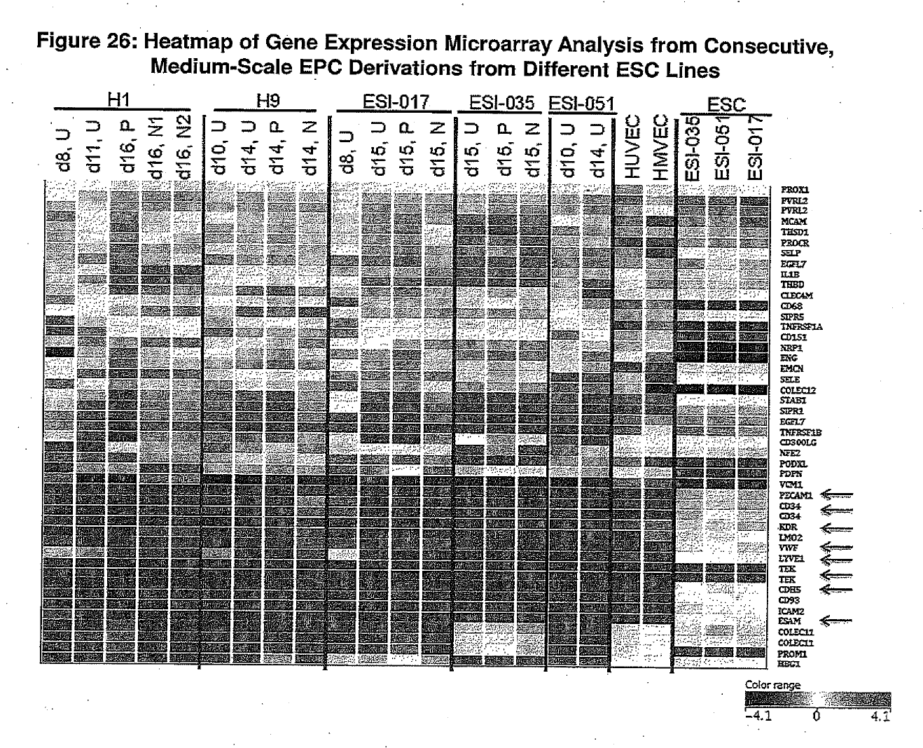

FIG. 26 shows a heatmap of gene expression from medium-scale EPC derivations from different ESC lines. Total RNA was extracted from cells using Qiagen RNeasy mini kits. RNA concentrations were measured using a Beckman DU530 or Nanodrop spectrophotometer, and RNA quality determined by denaturing agarose gel electrophoresis or an Agilent 2100 bioanalyzer. cRNA was hybridized to Illumina whole-genome HumanHT-12 v4 Expression BeadChips, and data was read using a BeadStation array reader (Illumina). Raw data was imported into Genespring GX 11.0 (Agilent), and percentile-shift normalized and log transformed. Hierarchical clustering on entities was carried out in Genespring GX 11.0 using a Euclidean distance metric. Red arrows at the lower right of the heatmap indicate several canonical endothelial markers (i.e., PECAM 1; CD34; KDR; VWF; LYVE1; TEK; CDH5; and ESAM).

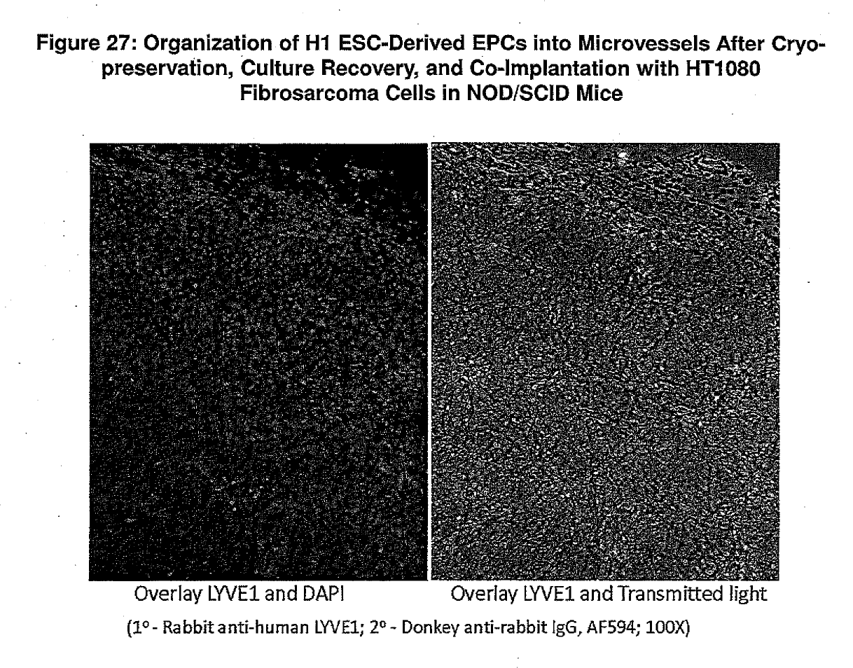

FIG. 27 shows that H1-derived EPCs develop into microvessels in an in vivo model system. H1 ESC-derived EPCs produced as described above were recovered from cryostorage and briefly expanded in culture, then co-injected with HT1080 fibrosarcoma cells (1 and 3 million cells, respectively) sub-Q in NOD/SCID mice. After 11-13 days the tumors were excised, fixed in formalin and paraffin-embedded, then thin-sectioned and stained for immunofluorescence microscopy following antigen retrieval. FIG. 27 shows results for human LYVE-1 staining.

### Example 4

### Molecular Profile of hES Derived Endothelial Cells

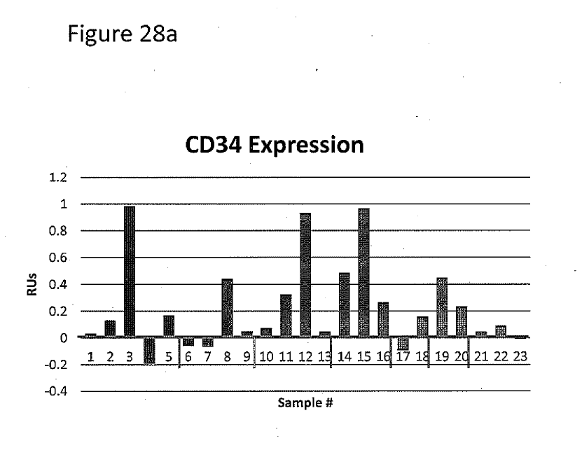

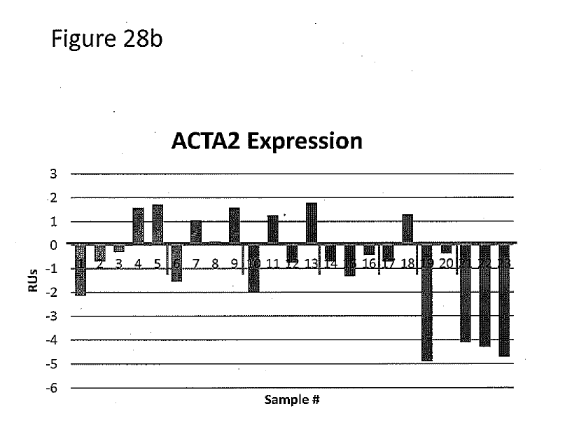

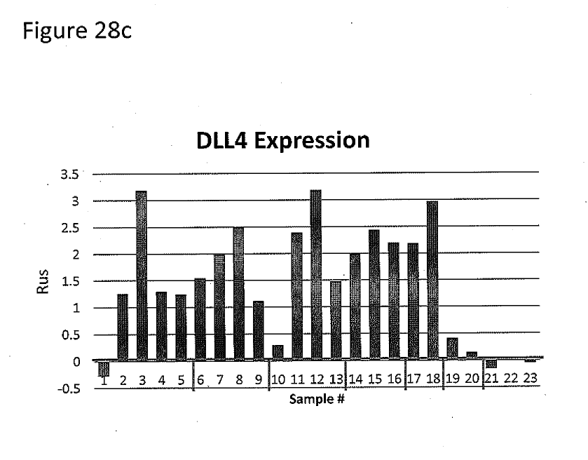

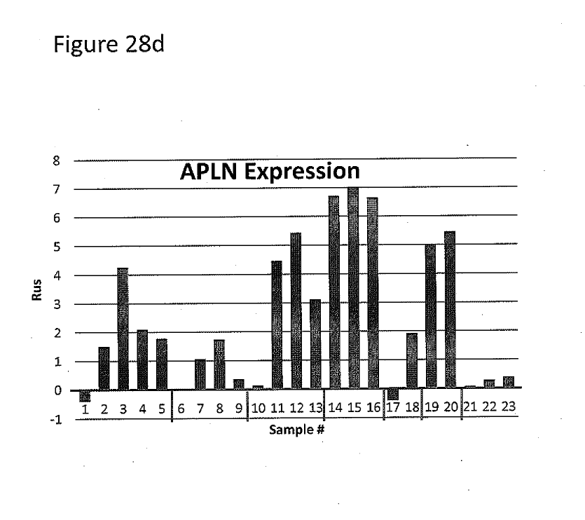

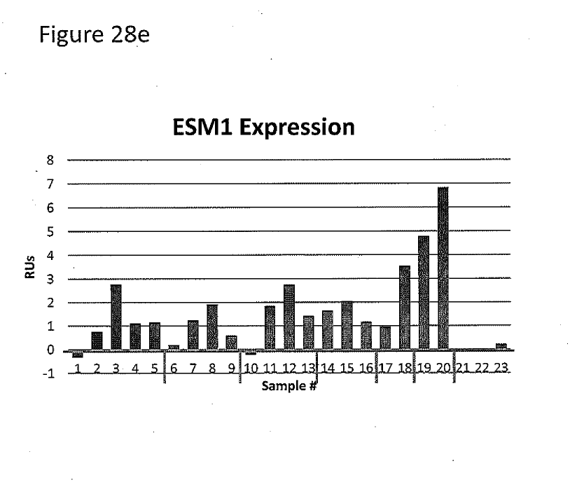

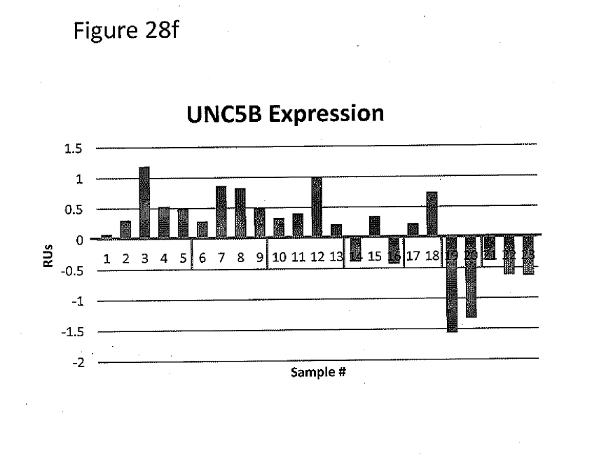

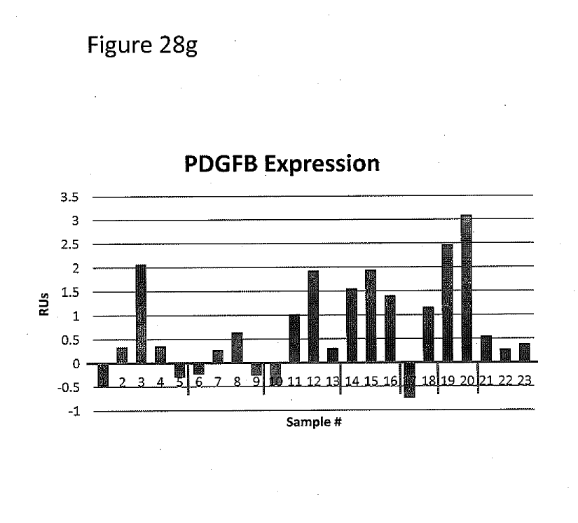

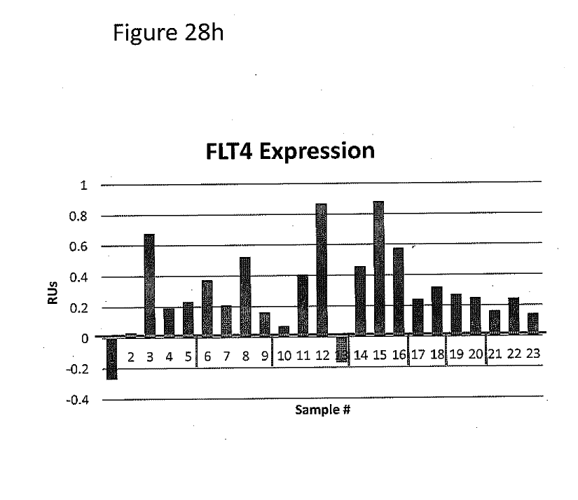

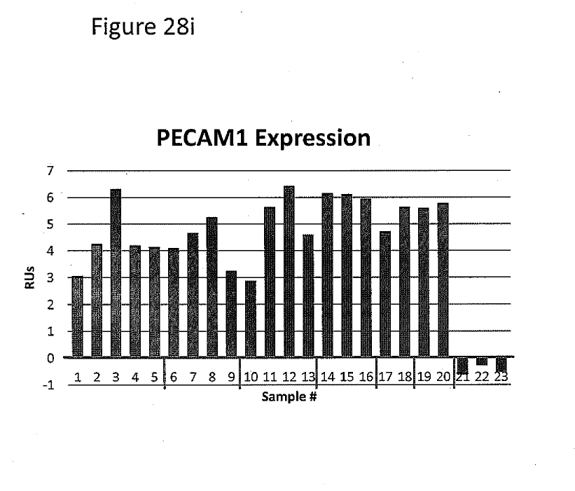

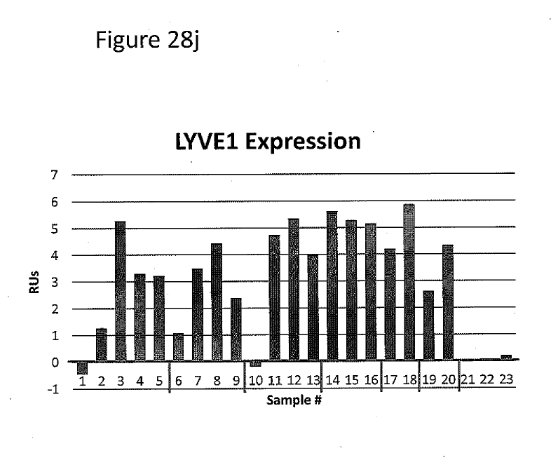

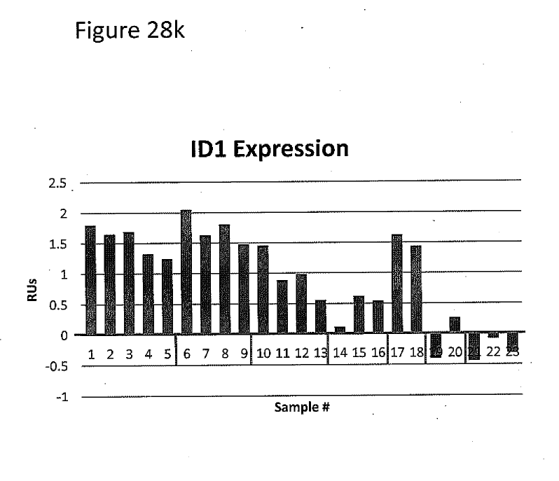

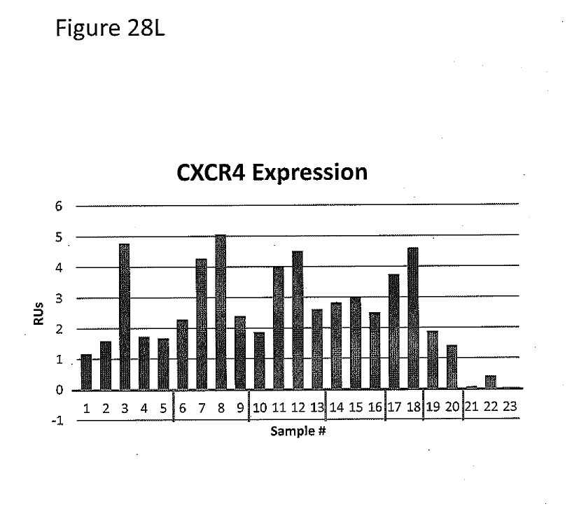

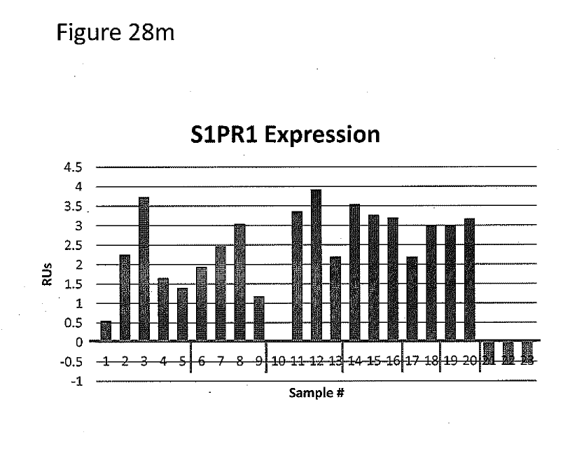

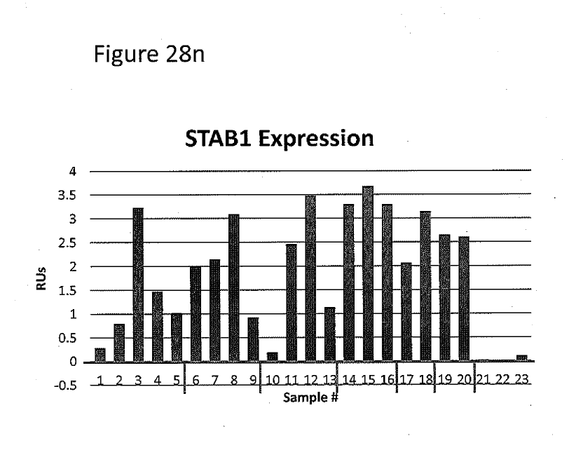

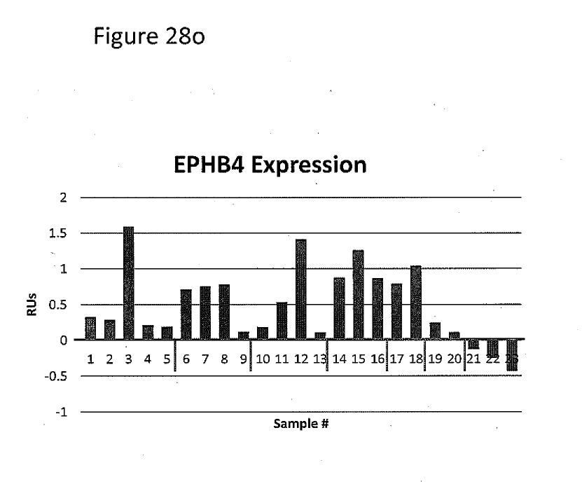

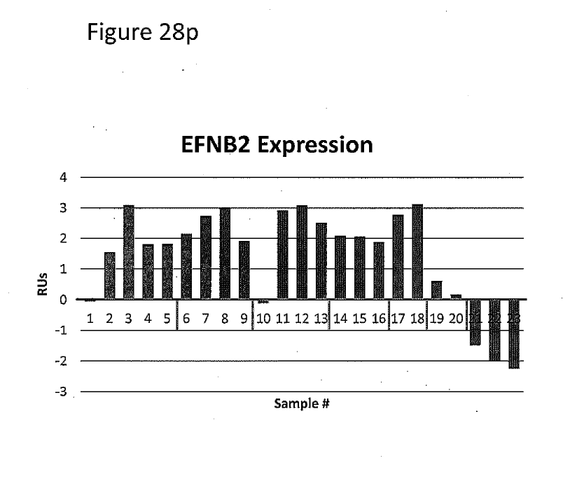

Endothelial cells produced according to the methods described infra were analyzed for expression of endothelial cell (EC) associated gene expression. Molecular profiling of human embryonic stem cell (hESC)-derived endothelial cells (ECs), primary ECs, and undifferentiated hESC lines was performed as follows. Total RNA was extracted from cells using Qiagen RNeasy mini kits according to the manufacturer's instructions. RNA concentrations were measured using a Beckman DU530 or Nanodrop spectrophotometer, and RNA quality determined by denaturing agarose gel electrophoresis or an Agilent 2100 bioanalyzer. cRNA was hybridized to Illumina whole-genome HumanHT-12 v4 Expression BeadChips, and data was read using a BeadStation array reader (Illumina). Raw data was imported into Genespring GX 11.0 (Agilent), percentile-shift normalized and log-transformed. Data for a selection of EC-associated genes are shown as bar histograms of log-transformed resonance units (RUs) in FIG. 28 a-p. FIG. 28a shows CD34 expression; FIG. 28b shows ACTA2 expression; FIG. 28c shows DLL4 expression; FIG. 28d shows APLN expression; FIG. 28E shows ESM1 expression FIG. 28F shows UNC5B expression; FIG. 28g shows PDGFB expression; FIG. 28h shows FLT4 expression; FIG. 28i shows PECAM1 expression; FIG. 28j shows LYVE1 expression; FIG. 28k shows ID1expression; FIG. 28l shows CXCR4 expression; FIG. 28m shows S1PR1 expression; FIG. 28n shows STAB 1 expression; FIG. 28o shows EPHB4 expression; FIG. 28p shows EFNB2 expression.

Key to sample #s is as follows: Sample #s 1-5, ECs derived from hESC line H1 (WA01); #s 6-9, ECs derived from hESC line H9 (WA09); #s 10-13, ECs derived from hESC line ESI-017; #s 14-16, ECs derived from hESC line ESI-035; #s 17-18, ECs derived from hESC line ESI-051; #19, human umbilical vein endothelial cells (HUVECs) passage 6; #20, human microvascular endothelial cells (HMVECs) passage 6; #21, undifferentiated hESC line ESI-035 passage 25; #22, undifferentiated hESC line ESI-051 passage 23; #23, undifferentiated hESC line ESI-017 passage 24. Sample #1, EC differentiation cultures harvested on day 8; #2, EC differentiation cultures harvested on day 11; #3, EC differentiation cultures harvested on day 16 CD34-positive cell fraction; #4, EC differentiation cultures harvested on day 16 CD34-depleted fraction; #5, EC differentiation cultures harvested on day 16 residual CD34-positive cells removed from CD34-depleted fraction (follows #3 and precedes #4); #6, EC differentiation cultures harvested on day 10; #7, EC differentiation cultures harvested on day 14 (unseparated); #8, EC differentiation cultures harvested on day 14 CD34-positive fraction; #9, EC differentiation cultures harvested on day 14 CD34-depleted fraction; #10, EC differentiation cultures harvested on day 8; #11, EC differentiation cultures harvested on day 15 (unseparated); #12, EC differentiation cultures harvested on day 15 CD34-positive fraction; #13, EC differentiation cultures harvested on day 15 CD34 depleted fraction; #14, EC differentiation cultures harvested on day 15 (unseparated); #15, EC differentiation cultures harvested on day 15 CD34-positive fraction; #16, EC differentiation cultures harvested on day 15 CD34-depleted fraction; #17, EC differentiation cultures harvested on day 10; #18, EC differentiation cultures harvested on day 14 (unseparated). Immunomagnetic selections for CD34-expressing cells were performed using the Dynal CD34 Progenitor Cell Selection System (Invitrogen) for H1 hESC-derived ECs and CD34 MACS MicroBead kits (Miltenyi Biotec) for H9, ESI-017, and ESI-035 hESC-derived ECs both according to the manufacturer's instructions.

A number of findings from these studies are noteworthy, including but not limited to the effective removal of α-smooth muscle actin (ACTA2)-expression in the CD34-positive cell immunoselected fractions, the upregulated expression of endothelial tip cell-associated genetic markers (such as DLL4, APLN, ESM1, UNC5B) in the CD34-positive cells (see, e.g., del Toro et al. (2010) Blood 116:4025-4033; Geudens and Gerhardt (2011) Development 138:4569-4583), the upregulated expression of lymphatic endothelial progenitor/precursor cell-associated genes (such as FLT4/VEGFR3, LYVE1, CD133) in the CD34-positive cell fractions (see, e.g., Salven et al. (2003) Blood 101:168-172), the upregulated expression in the CD34-positive cell fractions of genes for the receptor tyrosine kinase EPHB4 which was reported elsewhere to be undetectable in hESC-derived ECs (see, e.g., James et al. (2010) Nat. Biotechnol. 28:161-166), and upregulated expression in the CD34-positive cell fractions of genes for both EPHB4 receptor and its ligand ephrinB2 (EFNB2) which were reported elsewhere to be differentially expressed in venous and arterial endothelium, respectively (see, e.g., Swift and Weinstein (2009) Circ. Research 104:428-441).

### Example 5

### FACS Analysis of Cryopreserved EC

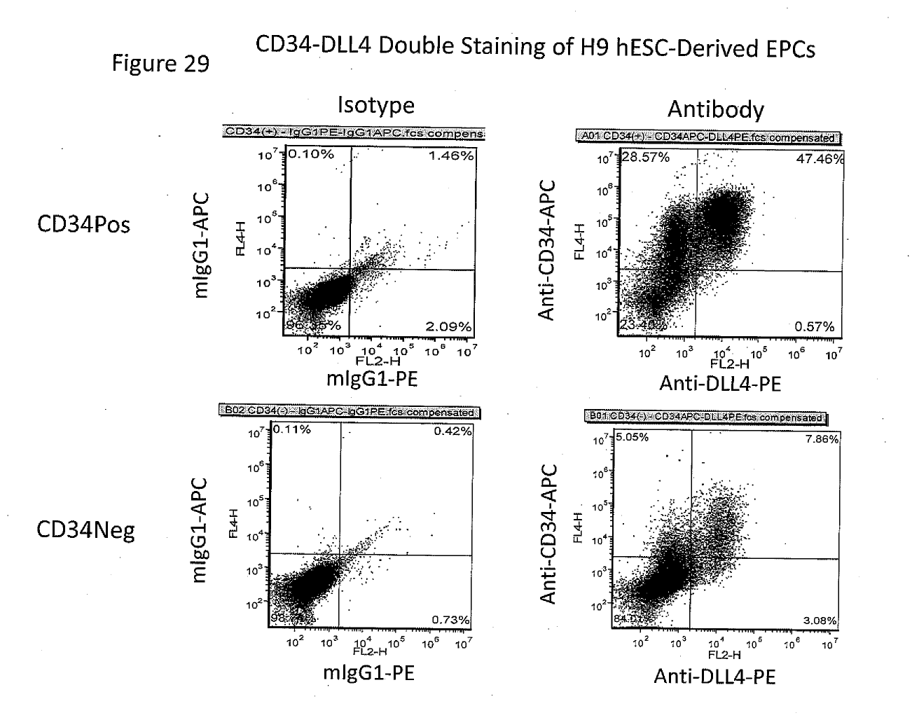

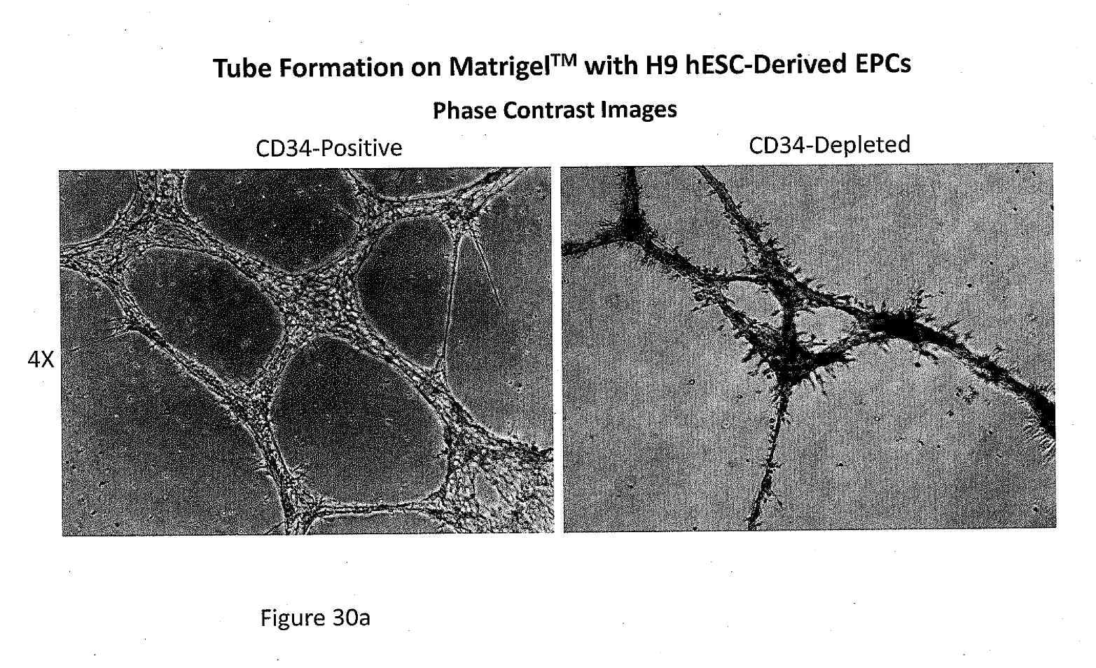

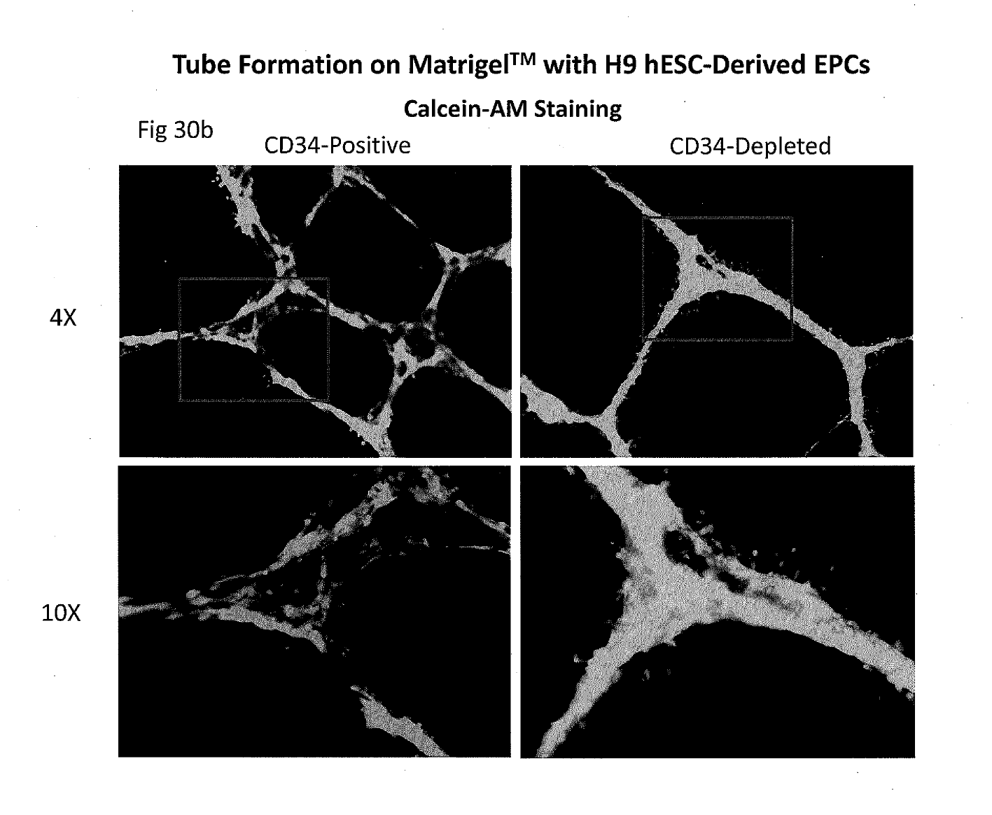

This experiment analyzed co-expression of Delta-like ligand 4 (DLL4) with CD34 on hESC-derived EPCs by flow cytometry analysis. Cryopreserved immunoselected CD34-positive and CD34-depleted cells from day 14 H9 hESC-derived EPC cultures (corresponding to sample #s 8 and 9, respectively, in the preceding example 4) were thawed and cultured overnight in Stemline medium with stage 2 factors (as disclosed infra) on human fibronectin-coated tissue culture flasks at 37° C. The cells were then harvested using Accutase and double-immunostained using antibodies to DLL4 (PE-conjugated, BioLegend) and CD34 (APC-conjugated, BioLegend) or the respective PE- and APC-conjugated Ig isotype controls, and were analyzed using an Accuri C6 flow cytometer. The results, shown in FIG. 29, confirmed the expression of DLL4 protein and its strong association with the immunoselected EPC positive fraction. (after cryopreservation to full recovery of CD34 expression levels (shown in earlier examples) was seen after culturing the cells for about 3 days).

### Example 6

### Endothelial Cell Tube Formation Assay

The spontaneous formation of tubular or capillary-like structures by hESC-derived EPCs on Becton Dickinson Matrigel basement membrane matrix in vitro was used to assess angiogenic potential. Cryopreserved immunoselected CD34-positive and CD34-depleted cells from day 14 H9 hESC-derived EPC cultures (corresponding to sample #s 8 and 9, respectively, in Example 4) were thawed and cultured overnight in Stemline medium with stage 2 factors on human fibronectin-coated TC flasks at 37° C. The cells were then harvested using Accutase, resuspended in Endothelial Growth Medium MV2 (PromoCell), plated in Matrigel (10 mg/ml, BD #354234)-coated 24-well plates and cultured overnight in a 37° C. incubator according to the standard assay protocol furnished by the supplier. Following this incubation, the endothelial tubes were labeled with BD Calcein AM fluorescent dye, following again instructions in the BD protocol. The cultures were then observed and photographed using phase contrast and fluorescence microscopy. Results showed clearly superior tube formation by the CD34-positive immunoselected cells compared to the CD34-depleted cells. The former cells produced robust multinodal networks with tubules interconnecting aggregates of endothelial cells and with the appearance of well-developed lumens. By contrast, the CD34-depleted cells produced fewer and less developed tubules with fewer nodes, and appeared to have an impaired ability overall to migrate and form the tubules in a controlled or coordinated manner.

### CONCLUSIONS

As shown in the Examples provided above, the present invention provides significant benefits over EPC production protocols currently in use. We established an industrialized scalable and consistent process for the derivation, expansion and banking of EPCs from a panel of human ESC lines, including members of our proprietary bank of cGMP-compliant ESC lines.

Our production process uses entirely chemically defined, serum-free, xenogeneic component-free culture conditions.

The present invention can yield clinically useful numbers EPCs in relatively short time periods (i.e., is rapid and highly efficient). Specifically, the EPC production process described herein consistently yields 4−8×10⁸ cells (largest scale attempted to date) within 14-16 days that are >60-99% CD31/PECAM1-positive by FACS analysis.

The percentage of CD31 positive EPCs produced using the present invention is increased to 60-99% without the aid of a cell purification step (e.g., by FACS sorting or immunomagnetic selection) compared to 2-5% in current state of the art protocols (see, e.g., James et al. (2010) Nature Biotechnol. 28(2): 161-166 and Ferreira et al. (2007) Circ. Res. 101:286-294).

The co-expression of CD34 antigen on most or all CD31 positive EPCs facilitates the use of currently existing and clinically validated positive selection devices for CD34 already employed for hematopoietic stem and progenitor cell purification and transplantation, thereby expediting the transition of EPCs of the present invention for clinical uses.

Both unselected and CD34-immunoselected day 14-16 EPCs can be cryopreserved (serum-free) and efficiently recovered in further expansion cultures with retention of their antigenic phenotype and basic functional properties in vitro and in vivo.

A further benefit of the present invention is that it can yield bi-potent EPCs, i.e., EPCs that express markers of both blood vascular endothelial (e.g., PV-1/PAL-E, PLVAP, plasmalemma vesicle-associated protein 1) and lymphatic endothelial cells (e.g., LYVE-1). This increases the options for utilizing the cells therapeutically, such as for repairing sites of vascular injury or for targeting sites of tumor angiogenesis (or neovascularization) or metastasis through co-opted lymphatic vessels. PV-1(PAL-E) and LYVE-1 are co-expressed on CD34-positive cells (and with each other) at day 14-16 of derivation.

The results of the present invention are unlike those obtained using a current state of the art process (see James et al. (2010) ibid.). Microarray analysis in James et al. indicate less than a 0.5-fold increase in CD34 RNA expression over background in unpurified day 14 endothelial cell cultures (Phase 1-derived cells). Using this process, a further increase of 10-fold or more in CD34 RNA expression was observed only as the result of an isolation (i.e., purification) step that increased the CD31-expressing endothelial cells to more than 95%. Also, James et al. stated that Phase 1-derived cells do not show increased levels of factors typical of lymphatic endothelial cells.

The results of the present invention are also unlike those obtained in Ferreira et al. As described previously, the culture system in this reference contained numerous materials of xenogeneic origin, including serum or bovine albumin and could reasonably be expected to undergo substantial experimentation and substitutions before it could be translated into clinical therapies. Moreover, this process is comparatively lengthy, requiring around 28 days to complete. Moreover, CD34 and CD31 (PECAM1) antigen-expressing cells differentiated from H9 ES cells were 65% and 98% positive, respectively, and from H13 ES cells were 14% and 39% positive, respectively.

By comparison, CD34 antigen-expressing cells using the process of the present invention were shown to comprise more than 90%, usually 96-99%, of the cells collected from the third passage at 13 days following seeding from embryoid bodies (18 days from initiation of ES cell culture to form embryoid bodies) in all 3 ES cell lines tested. In addition, the process according to the present invention is scalable for producing at least hundreds of millions of EPCs. This significant increase in demonstrated scalability which is not known to have been reported in current state of the art protocols makes the present invention suitable for generating therapeutic amounts of EPCs that can be employed in any of a variety of treatments.

Aspects of the present invention achieve these increases in EPC production by employing one or more of the following: generating uniform EBs; eliminating serum from the EPC generation protocol; using xenogenic component-free conditions in the EPC generating protocol; employing a chemically defined culture system.

In addition, the EPCs generated according to aspects of the present invention are true endothelial progenitor/precursor cells (EPCs) and not committed endothelial cells (ECs), as exemplified by the presence in the population of cells that have stable co-expression of CD34 and CD31 as well as cells that have stable co-expression of LYVE-1 (a lymphatic vessel endothelial cell marker) and PV-1/PAL-E (a blood vascular endothelial cell marker).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of differentiating embryonic stem cells into an endothelial progenitor cell comprising a) framing an embryoid body from the embryonic stem cells; b) culturing the embryoid body; c) contacting the embryoid body with a differentiation cocktail comprising BMP4 and optionally comprising activin; d) contacting the embryoid body of c) with a differentiation cocktail comprising activin and BMP4; e) contacting the embryoid body of d) with FGF2; f) transferring the embryoid body of e) to an adherent cell culture vessel so as to form a cell monolayer; g) contacting the cell monolayer of f) with differentiation cocktail comprising BMP4, FGF2 and VEGF; h) contacting the cell monolayer of g) with a TGF-β inhibitor and a cell culture media lacking exogenously added BMP4; i) culturing the monolayer of g) for a sufficient period of time to obtain endothelial progenitor cells, thereby differentiating embryonic stem cells into an endothelial progenitor cell.

2. The method of claim 1, wherein after step c) and before step d) the embryoid body is transferred to a low attachment tissue culture vessel.

3. The method of claim 1, wherein the embryonic stem cells are human embryonic stem cells.

4. The method of claim 1, wherein the adherent culture vessel comprises a matrix.

5. The method of claim 4, wherein the matrix is fibronectin.

6. The method of claim 1, wherein the TGF-β inhibitor is SB431542.

7. The method of claim 1, wherein the embryoid body is cultured for about a day in step b).

8. A proliferating cell that expresses both LYVE-1 and PV-1PAL-E.

9. The proliferating cell of claim 8, wherein the cell is a human cell.

10. The proliferating cell of claim 8, wherein the proliferating cell has essentially the same genome as a human embryonic stem cell line.

11. The proliferating cell of claim 8, wherein the cell can differentiate into a vascular endothelial cell.

12. The proliferating cell of claim 8, wherein the cell can differentiate into a lymphatic endothelial cell.

13. A system for making endothelial cells comprising a first population of cells comprising stem cells and a second population of cells comprising bipotential endothelial progenitor cells.

14. The system of claim 13, wherein stein cells are embryonic stem cells.

15. The system of claim 13, wherein the stem cells are induced pluripotent cells.

16. The system of claim 13, wherein the bipotential endothelial progenitor cells express both LYVE-1 and PV-1PAL-E.

17. The system of claim 13, wherein the second population of cells comprising bipotential endothelial progenitor cells comprises cells expressing LYVE-1 and cells expressing PV-1PAL-E.

18. The system of claim 13, wherein the first and second cell populations are contained in the same container.

19. The system of claim 13, wherein the first and second cell populations are contained in separate containers.
